(19) United States
(12) Patent Application Publication
(10) Pub. No.: US 2009/0048535 A1
(43) Pub. Date: Feb. 19, 2009

(54) Detecting Cross-contamination in Blood Measurements with a Multilumen Catheter

(76) Inventors: Mark Ries Robinson, Albuquerque, NM (US); Mike Borrello, Carlsbad, CA (US); John O'Mahony, Maple Grove, MN (US); Richard Thompson, Dana Point, CA (US); V. Gerald Grafe, Corrales, NM (US)

Correspondence Address: V. Gerald Grafe, esq., P.O. Box 2689, Corrales, NM, 87048, US

(21) Appl. No.: 11/860,545

(22) Filed: Sep. 25, 2007

Related U.S. Application Data
(60) Provisional application No. 60/955,636, filed on Aug. 13, 2007.

(51) Int. Cl. A61B 5/155

(52) U.S. Cl. 600/581; 128/898

(57) ABSTRACT

The present invention comprises methods and apparatuses that can provide accurate measurement of glucose or other analytes from a multilumen catheter in the presence of infusion of substances, including glucose. Examples of “multilumen catheters” include central venous catheters having multiple lumens, midline catheters having multiple lumens, multiple catheters configured or emplaced such that their lumens are in proximity to each other, and, in the case of indwelling analyte sensors, a catheter with a lumen for infusion and an indwelling sensor spaced apart from the infusion lumen. For blood withdrawal, anti-cross contamination controls can prevent the entrainment of blood which might be contaminated with feeding fluids or medications that are administered through other lumens within the catheter and in proximity of the blood sampling port. Cross contamination can occur under various situations, and is known to occur when the patient is connected to a ventilator. The ventilator cyclically raises the intra-thoracic pressure and diminishes blood flow rate in the central veins returning to the heart. The diminished flow can increase the chances for cross-contamination when additional lumens are introducing fluids during a draw sample.

### CROSS-REFERENCE TO RELATED APPLICATIONS g

This application claims the benefit of U.S. provisional application 60/955,636, filed Aug. 13, 2007, which is incorporated herein by reference. This application is related to U.S. provisional application 60/791,719, filed Apr. 12, 2006, and to U.S. provisional application 60/737,254, filed Nov. 15, 2006, each of which is incorporated herein by reference.

### FIELD OF THE INVENTION

This invention relates to the field of the measurement of blood sampling, and more specifically to management of cross-contamination when sampling blood from a central venous catheter in the presence of infusion in the same catheter.

### BACKGROUND OF THE INVENTION

Tight glycemic control. Many peer-reviewed publications have demonstrated that tight control of blood glucose significantly improves critical care patient outcomes. Tight glycemic control (TGC) has been shown to reduce surgical site infections by 60% in cardiothoracic surgery patients and reduce overall ICU mortality by 40% with significant reductions in ICU morbidity and length of stay. See, e.g., Furnary Tony, Oral presentation at 2005 ADA annual, session titled “Management of the Hospitalized Hyperglycemic Patient;” Van den Berghe et al., NEJM 2001; 345:1359. Historically, caregivers have treated hyperglycemia (high blood glucose) only when glucose levels exceeded 220 mg/dl. Based upon recent clinical findings, however, experts now recommend IV insulin administration to control blood glucose to within the normoglycemic range (80-110 mg/dl). Adherence to such strict glucose control regimens requires near-continuous monitoring of blood glucose and frequent adjustment of insulin infusion to achieve normoglycemia while avoiding risk of hypoglycemia (low blood glucose). In response to the demonstrated clinical benefit, approximately 50% of US hospitals have adopted some form of tight glycemic control with an additional 23% expected to adopt protocols within the next 12 months. Furthermore, 36% of hospitals already using glycemic management protocols in their ICUs plan to expand the practice to other units and 40% of hospitals that have near-term plans to adopt TGC protocols in the ICU also plan to do so in other areas of the hospital.

Sampling from a central venous catheter. The effective implementation of tight glycemic control protocols generally requires the frequent measurement of glucose. This measurement process typically requires the procurement of a blood sample that is representative of the patient's physiological status. Samples can be obtained from a variety of means, including without limitation peripheral IV's, arterial blood lines, midline catheters peripherally inserted central catheters, and central venous catheters. Central venous catheters can be a preferred means of access due to the frequency of use in the ICU and the ability to make blood withdrawals on a regular basis. Most central venous catheters are multi-lumen catheters with the number of lumens being selected based upon patient needs. Catheters are referred to as monoluminal, biluminal or triluminal, dependent on the actual number of tubes or lumens (1, 2 and 3 respectively,). Some catheters have 4 or 5 lumens, depending on the reason for their use. The termination of the lumen in the body occurs at different locations. The termination point is typically referred to as a port. In the case of a multi-lumen catheter the port at the end of the catheter is defined as the distal port, with intervening ports referred to as medial ports and the port closest to the insertion into the body referred to as the proximal port. The catheter is usually held in place by a suture or staple and an occlusive dressing. Regular flushing with saline or a heparin-containing solution is performed to keep the line patent and prevent infection.

Central venous blood samples can be obtained through a variety of catheter types including a central venous catheter. Central venous catheters are utilized for many purposes to include drug infusion as well as blood sampling. When central venous catheters are utilized for procurement of a blood draw, nursing standards are very specific with respect to the procedure to be used. These standards require that all IV infusions be stopped and recommend a one minute wait time before drawing blood from the catheter. The rationale for both the stoppage and waiting period is to allow IV fluids and medications to be carried away from the catheter location such that the blood sample is not contaminated by the fluids being infused (the “infusate”). The mixing of IV fluids or medications in the blood sample is generally referred to as cross-contamination. Cross-contamination is the general process by which fluids being infused into the patient become present in the blood sample and can contaminate resulting measurements. FIG. 1 is a schematic illustration of the terms involved. A central vein 101 has disposed within it a multi-lumen catheter 102, and normal blood flow from left to right in the figure at a rate denoted FR. The catheter 102 has a first port 103 from which it is desired that a sample be withdrawn at a withdrawal rate denoted WR. The catheter 102 has a second port 104 through which an infusate is infused into the vessel at a rate denoted IR.

Although central venous catheters can be placed in a variety of locations, the typical placement is to have the tip 3-4 cm above the entrance to the right atrium. This places the tip in the center of the superior vena cava and the proximal opening about 6 cm back from the tip. The proximal port will typically be in the vein where the device was introduced; i.e. the brachial cephalic or internal jugular vein. The flow characteristics surrounding the ports of the central venous catheter can have direct influence on the possibility of cross-contamination. The superior vena cava is the main vein for the drainage of the superior aspect of the body. It is about 7 cm in length and is formed by the confluence of the brachiocephalic veins. It has no valves and ends in the right atrium. It is approximately 20 mm in diameter. The inferior vena cava has similar flow characteristics but the flow rates are strongly dependent upon exercise involving the lower extremity. Flow in the central vena cava is variable and is affected by the cardiac cycle and respiration. FIG. 2 is an illustration of a typical tracing of the flow rates as a function of the cardiac cycle. In normal physiology, peak flow is during systole and is 30-45 cm/sec. At the beginning of the cardiac cycle, the flow rate is zero or slightly negative. There is a brief period of retrograde flow as the right ventricle contracts and it takes a finite amount of time for the valve to shut. Furthermore the valve tends to push out into the right atrium as the ventricle contracts.

Difficulties in tight glycemic control when using a central venous catheter. For blood glucose measurement systems that utilize a central venous access catheter for procurement of a blood sample for subsequent analysis or place a catheter in the superior or inferior vena cava, the potential impact of cross-contamination involving a glucose containing fluid can be quite dramatic. For example, if the patient is being infused with a 5% dextrose solution (5000 mg/dl), and 1% cross-contamination occurs, the measured glucose value can be in error by 50 mg/dl. Given that the typical target range for tight glycemic control is between 80 and 120 mg/dl, a potential over-estimation by 50 mg/dl can have serious consequences. As an example, the patient might be given additional insulin due to the inaccurately high glucose measurement result. The actual overall systemic glucose would be consequently decreased while the measured glucose might remain high due to the presence of glucose via cross-contamination. Cross-contamination with non-glucose containing fluids also can affect the measurement, but are typically less significant since they generally result in a decreased glucose measurement. The impact is simply volumetric so at a glucose value of 100 mg/dl a 10% dilution can result in a glucose measurement of 90 mg/dl, and such slightly low glucose readings are less likely to have such dramatic undesirable treatment errors.

Accordingly, there is a need for methods and apparatuses that allow accurate glucose measurements from catheters, especially central venous catheters, in the presence of infusion of substances including glucose.

### SUMMARY OF THE INVENTION

The present invention comprises methods and apparatuses that can provide accurate measurement of glucose or other analytes from a multilumen catheter in the presence of infusion of substances, including glucose. Examples of “multilumen catheters” include central venous catheters having multiple lumens, midline catheters having multiple lumens, multiple catheters configured or emplaced such that their lumens are in proximity to each other, and, in the case of indwelling analyte sensors, a catheter with a lumen for infusion and an indwelling sensor spaced apart from the infusion lumen. For blood withdrawal, anti-cross contamination controls can prevent the entrainment of blood which might be contaminated with feeding fluids or medications that are administered through other lumens within the catheter and in proximity of the blood sampling port. Cross contamination can occur under various situations, and is known to occur when the patient is connected to a ventilator. The ventilator cyclically raises the intra-thoracic pressure and diminishes blood flow rate in the central veins returning to the heart. The diminished flow can increase the chances for cross-contamination when additional lumens are introducing fluids during a draw sample.

### BRIEF DESCRIPTION OF THE DRAWINGS

FIG. 1 is a schematic illustration of terms relevant to the present invention.

FIG. 2 is an illustration of a typical tracing of the flow rates as a function of the cardiac cycle.

FIG. 3 is a schematic illustration of the laboratory system.

FIG. 4 is a schematic depiction of three blood flow velocity profiles investigated in an experiment related to the present invention.

FIG. 5 is a schematic illustration of sample contamination in an experiment related to the present invention.

FIG. 6 is a schematic illustration of the placement of the catheter and the orientation of the proximal port in an experiment related to the present invention.

FIG. 7 is an illustration of a test circuit and test procedure related to the present invention.

FIG. 8 is an illustration of a test circuit used in an experiment related to the present invention.

FIG. 9 is an illustration of glucose level as a function of time in an experiment related to the present invention.

FIG. 10 is a summary of parameters related to cross contamination.

FIG. 11 is an illustration of glucose level as a function of time in an experiment related to the present invention.

FIG. 12-19 are illustrations of experimental conditions and results.

FIG. 20 is an illustration of relationships between pressure and mechanical ventilation.

FIG. 21 is a schematic illustration of a blood access circuit used for demonstration of measurement instability due to cross-contamination.

FIG. 22 is an illustration of the overall stability of the measurement during the withdrawal period when the system is simply pulling blood from the beaker.

FIG. 23 is an illustration of the stability of the measurement when injecting a 60 microliter bolus but where the blood bolus has the same glucose concentration as the blood being withdrawn from the beaker.

FIG. 24 is an illustration of the stability of the measurement when injecting a 60 microliter bolus but where the blood bolus has a 2560 mg/dl glucose concentration.

FIG. 25 is an illustration of the stability of the measurement when injecting a 60 microliter bolus but where the blood bolus has a 1240 mg/dl glucose concentration.

FIG. 26 is a schematic illustration of a blood access used in connection with the present invention.

FIG. 27 is an illustration of pressure tracing obtained during eight automated sample withdrawal, measurement, re-infusion and cleaning cycles.

FIG. 28 is an illustration of intravascular pressure changes due to ventilation.

FIG. 29 is a schematic illustration of a compliance isolation method according to the present invention.

FIG. 30 is an illustration of the simulated pressure and flow responses during a withdrawal where the compliance isolation method is used.

FIG. 31 is a schematic illustration of a flow feedback method, using a flow sensor in the blood line to sense fluid flow which can be compared to a desired flow.

FIG. 32 is an illustration of the operation of the flow feedback control method during a withdrawal.

FIG. 33 is a schematic block diagram of a cascade, pressure-flow control method according to the present invention.

FIG. 34 is an illustration of the operation of the cascade, pressure-flow control method.

FIG. 35 is a schematic block diagram of a pressure control method according to the present invention.

FIG. 36 is an illustration of the operation of the pressure control method.

FIG. 37 is an illustration of catheter flow with no active control.

FIG. 38 is an illustration of catheter flow with clamping or isolation compliance control.

FIG. 39 is an illustration of catheter flow with pressure control.

### DETAILED DESCRIPTION OF THE INVENTION

While nursing guidelines provide for operation that reduces the risk of cross-contamination, there is little if any published literature that has any evidence that cross-contamination actually occurs with central venous catheters. There are published reports of cross-contamination when using central venous catheters for dialysis but the withdrawal flow rate are in excess of 100 ml/min. Discussions with greater than 20 intensive care professions confirmed the practice of turning off infusion pumps prior to blood sample withdrawal but the mechanism by which cross-contamination occurs was not known within the group of professional interviewed.

A computational fluid dynamics investigation was performed to more fully understand the potential for cross-contamination. The investigation used reasonable variations of several variables to examine the potential for cross-contamination. Variables examined and varied within reasonable limits were normal physiology flow rates in both the inferior and superior vena cava, typical intravenous infusion rates of 5% dextrose solutions, typical catheter port separation distances, and blood withdrawal rates. The investigation concluded that under the conditions investigated there was no reasonable potential for cross-contamination to occur. An identified limitation of the investigation was the relationship of the port to the wall of the vessel. Specifically, if the catheter is resting in the bottom of the vessel and the port is located on the bottom of the catheter, the flow characteristics surrounding the port would be quite different than in the center of the vessel as modeled in the computational fluid dynamics investigation.

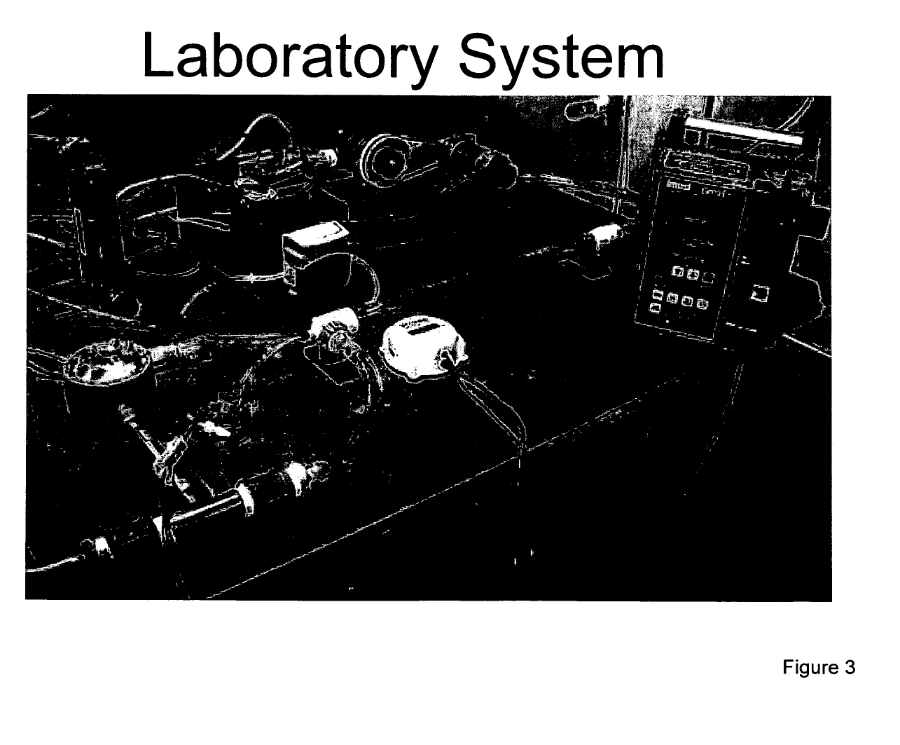

A laboratory experiment was performed to investigate variation in flow rates and variations in catheter orientation. FIG. 3 is a schematic illustration of the laboratory system. The laboratory system comprised a pump 301 capable of simulating velocity profiles in the vena cava, a Gemini infusion pump 302, a peristaltic withdrawal pump 303, an insertion type flow meter 304, a TDS conductivity meter 305, and the test section 306. The test section was constructed to simulate the superior vena cava (SVC), and is transparent acrylic with an internal diameter of 19.1 mm. The simulated blood flow travels through the flow meter 304, and enters the test section 306 through a 90 degree elbow 307, inducing turbulence in the fluid as it enters. The catheter 308 is inserted into the end of the elbow 307 and continues down inside the simulated SVC. The flow travels horizontally through the test section 306. The blood substitute is pumped from a source reservoir 309, and dumped into a sink reservoir 310 after it passes through the system. The infusion pump 302 injects either dye or potassium chloride solution into any desired catheter port, and the withdrawal pump 303 pulls fluid from any desired catheter port, through the TDS meter 305, and into the sink reservoir 310.

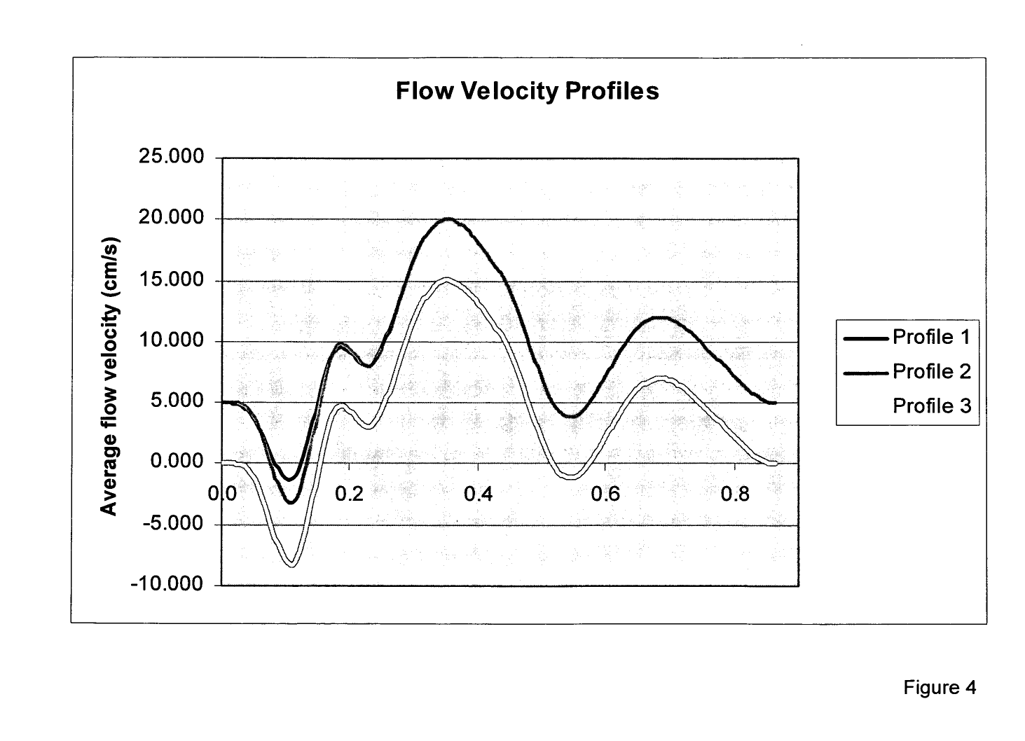

FIG. 4 is a schematic depiction of three blood flow velocity profiles investigated in the experiment. Profile 1 approximated a typical velocity profile in the SVC of a healthy adult. Profile 2 is similar to Profile 1, but with an exaggerated reverse flow region. Profile 3 was designed to encourage cross-contamination, and is similar to Profile 2 but with the velocity offset by −5 cm/sec throughout.

System verification. The conductivity meter was tested in both the installed and uninstalled conditions. While installed, it underreported the conductivity of the solutions sampled by a factor of 0.692. Because all of the conductivity measurements taken during testing were with the sensor installed in the system, the final cross-contamination values should not be affected. If desired, the true conductivity values can be obtained by multiplying all of the ppm readings by a factor of 1.45. All of the conductivity values presented in this description are the uncorrected numbers.

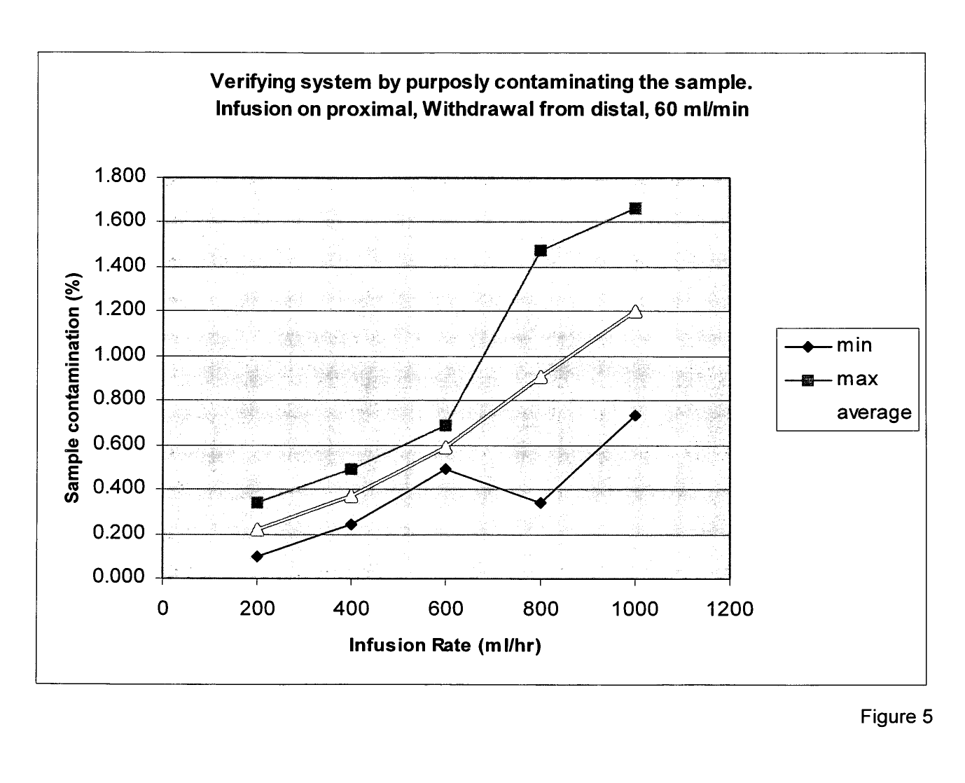

To verify that the system worked as intended, infusion and withdrawal ports were switched to purposely cause cross-contamination. A 3% potassium chloride solution was infused on the proximal port 321, and the sample was withdrawn from the distalport 322 at a rate of 60 ml/hr. Flow velocity profile 1 was used. The infusion rate was increased in steps of 200, 400, 600, 800, and 999 ml/hr. The results are presented in Table 1 and FIG. 5.

TABLE 1

| Infusion | | Venous | Infusion | Sample concentration | | | Cross-contamination | | |
|---|---|---|---|---|---|---|---|---|---|
| rate | Temp. | concentration | concentration | (PPM) | | | (%) | | |
| (ml/hr) | (deg C.) | (PPM) | (PPM) | Min | max | average | min | max | average |
| 200 | 27.1 | 430 | 20800 | 450 | 500 | 475 | 0.098 | 0.344 | 0.221 |
| 400 | 26.3 | 420 | 20800 | 470 | 520 | 495 | 0.245 | 0.491 | 0.368 |
| 600 | 26.3 | 420 | 20800 | 520 | 560 | 540 | 0.491 | 0.687 | 0.589 |
| 800 | 26.3 | 420 | 20800 | 490 | 720 | 605 | 0.343 | 1.472 | 0.908 |
| 999 | 22.8 | 410 | 20800 | 560 | 750 | 655 | 0.736 | 1.667 | 1.202 |

The verification data shows that sample contamination increases as infusion rate increases, verifying that the laboratory system works as expected when contamination is known to be present. The expanding range of minimum and maximum values might be due to turbulence caused by higher infusion rates. The percentage of cross-contamination was calculated using the following function:

$$Cross \text{-} contamination \% = \frac{( {conc}_{sample} - {conc}_{blood} )}{( {conc}_{infusion} - {conc}_{blood} )} \cdot 100$$

This function can also be used to calculate the minimum detectable level of cross-contamination. Inserting the measured concentrations of the simulated blood and infused fluid, and with the minimum detectable sample concentration rise of 10 ppm

$$Detectable cross \text{-} contamination = \frac{( 430 - 420 )}{( 20800 - 420 )} \cdot 100 = 0.049 \%$$

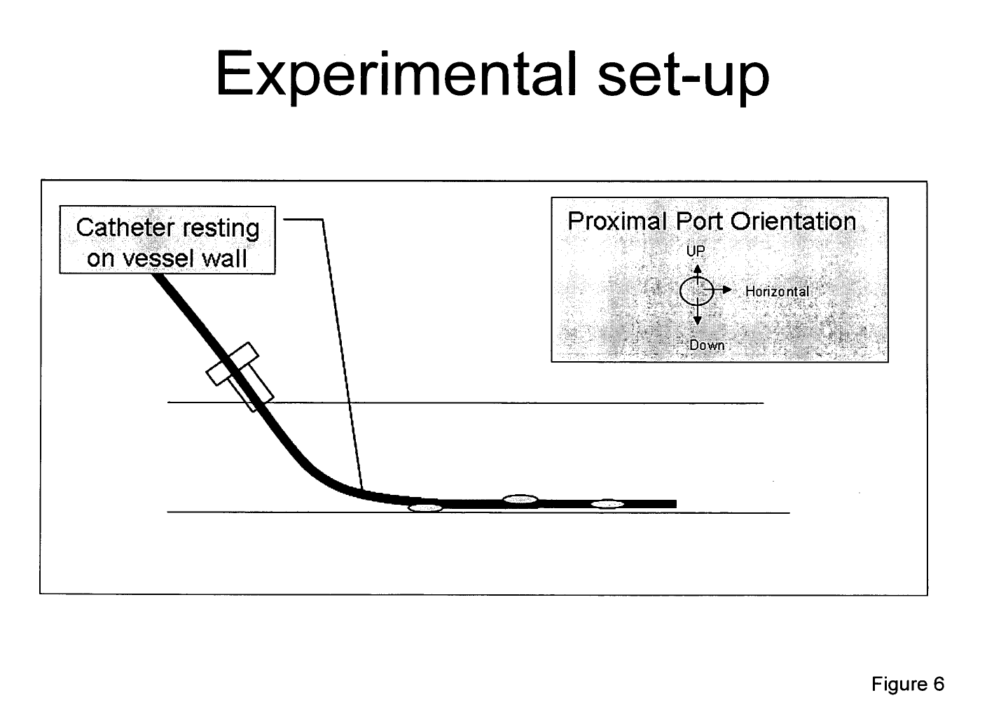

Experimental Design. Several sets of experiments were conducted to measure the cross-contamination during operation. The parameters were chosen in an attempt to increase cross-contamination as testing progressed. Infusion rates from 200 ml/hr to 999 ml/hr were tested. The concentration of the infused fluid was increased to 4% (uncorrected measurement of 27200 ppm) in order to increase the sensitivity in measured contamination levels. In addition, a test was performed with an Intralipid 20% solution consisting of about 10% potassium chloride (uncorrected measurement of 61000 ppm). FIG. 6 is a schematic illustration of the placement of the catheter and the orientation of the proximal port. The infusion rate was held constant at 500 ml/hour. Flow Profile 2 was used for all experiments.

Table 2 presents the results of the experiments with an infusion fluid of KCL and water.

TABLE 2

| Proximal Port | | | % Cross- |
|---|---|---|---|
| Orientation | Average Velocity | Withdrawal Rate | contamination |
| Down | 10 | 100 | 0.000 |
| Down | 4 | 100 | 0.000 |
| Up | 10 | 100 | 0.000 |
| Up | 4 | 100 | −0.015 |
| Horizontal | 10 | 100 | 0.015 |
| Horizontal | 4 | 100 | 0.000 |
| Down | 10 | 20 | 0.000 |
| Down | 4 | 20 | −0.015 |
| Up | 10 | 20 | 0.000 |
| Up | 4 | 20 | 0.015 |
| Horizontal | 10 | 20 | 0.015 |
| Horizontal | 4 | 20 | 0.000 |

Table 3 presents the results of the experiments with an infusion fluid of KCL and 20% Intralipid

TABLE 3

| Proximal Port | | | % Cross- |
|---|---|---|---|
| Orientation | Average Velocity | Withdrawal Rate | contamination |
| Down | 10 | 100 | 0.000 |
| Down | 4 | 100 | 0.000 |
| Up | 10 | 100 | 0.000 |
| Up | 4 | 100 | 0.000 |
| Horizontal | 10 | 100 | 0.000 |
| Horizontal | 4 | 100 | 0.000 |
| Down | 10 | 20 | 0.000 |
| Down | 4 | 20 | 0.000 |
| Up | 10 | 20 | 0.000 |
| Up | 4 | 20 | 0.000 |
| Horizontal | 10 | 20 | 0.000 |
| Horizontal | 4 | 20 | 0.000 |

There was no detectable cross-contamination during any of the tests. Calculating the minimum detectable contamination with the 4% (uncorrected measurement of 27200 ppm) solution, and assuming a detectable rise in 10 ppm, gives:

$$Detectable cross \text{-} contamination = \frac{( 420 - 410 )}{( 27200 - 410 )} \cdot 100 = 0.037 \%$$

And the minimum detectable contamination with the 10% (uncorrected measurement of 61000 ppm) solution gives:

$$Detectable cross \text{-} contamination = \frac{( 390 - 380 )}{( 61000 - 380 )} \cdot 100 = 0.017 \%$$

Therefore, the level of cross-contamination is below 0.037% in the KCl-water tests, and below 0.017% in the KCl-lntralipid testing. The laboratory testing demonstrated that the potential for cross-contamination is very low during typical use, and in experiments depicting cases worse than the typical operating conditions, cross-contamination was less than the detectable level of 0.017%.

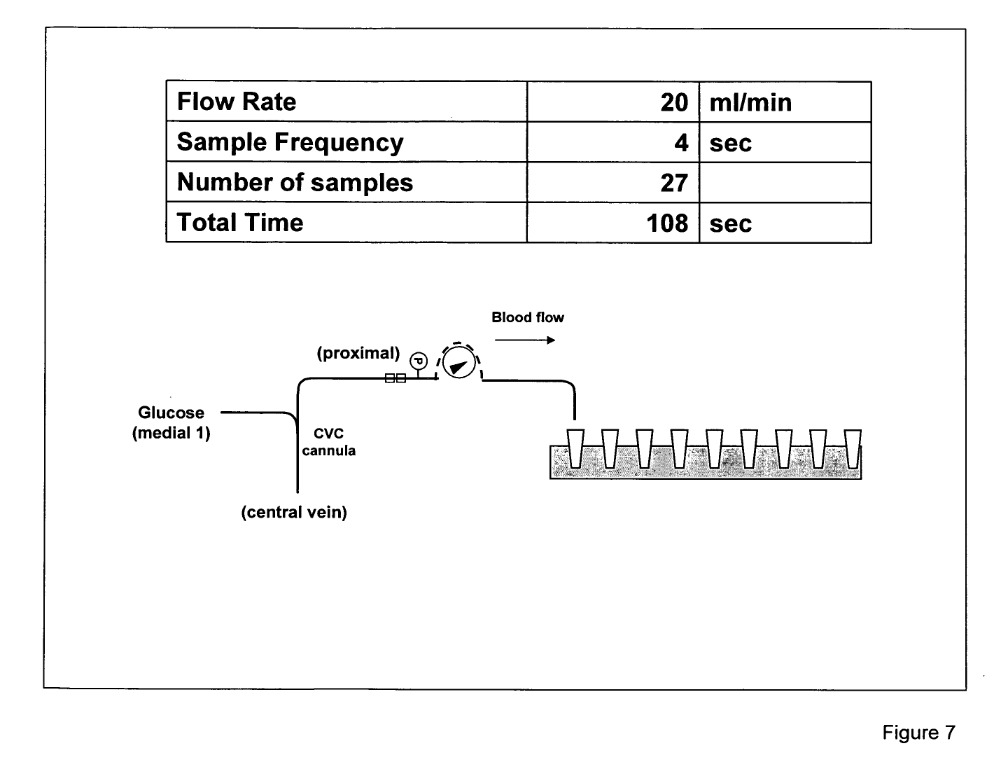

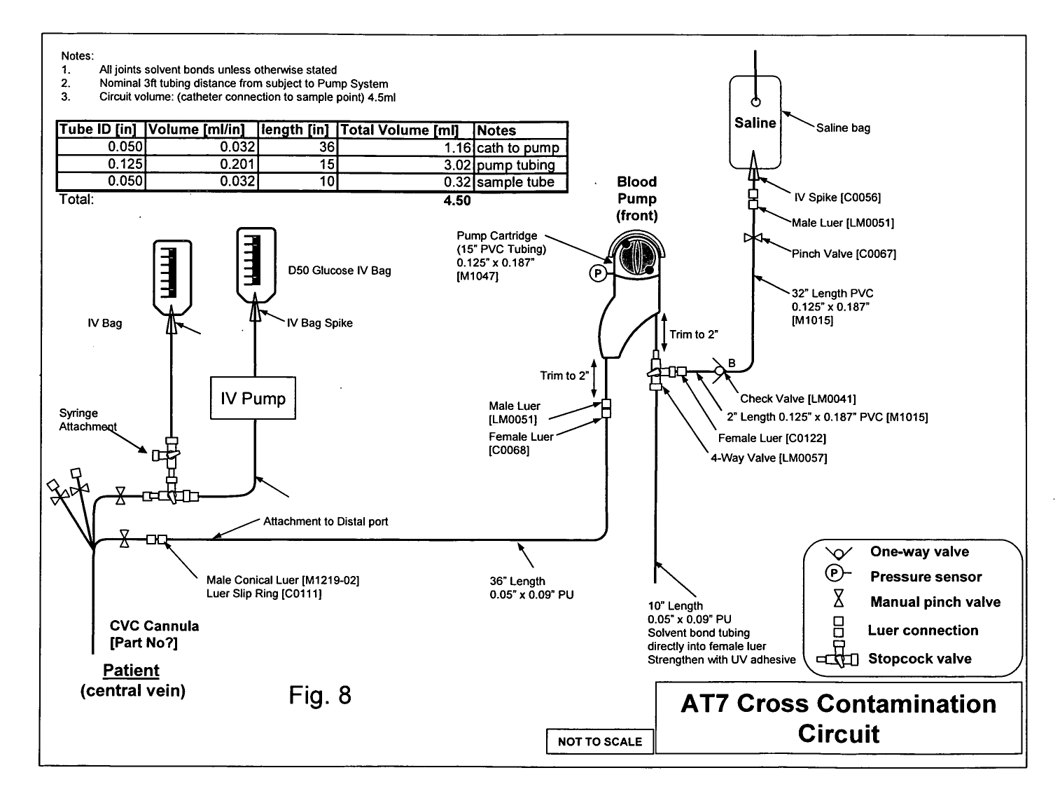

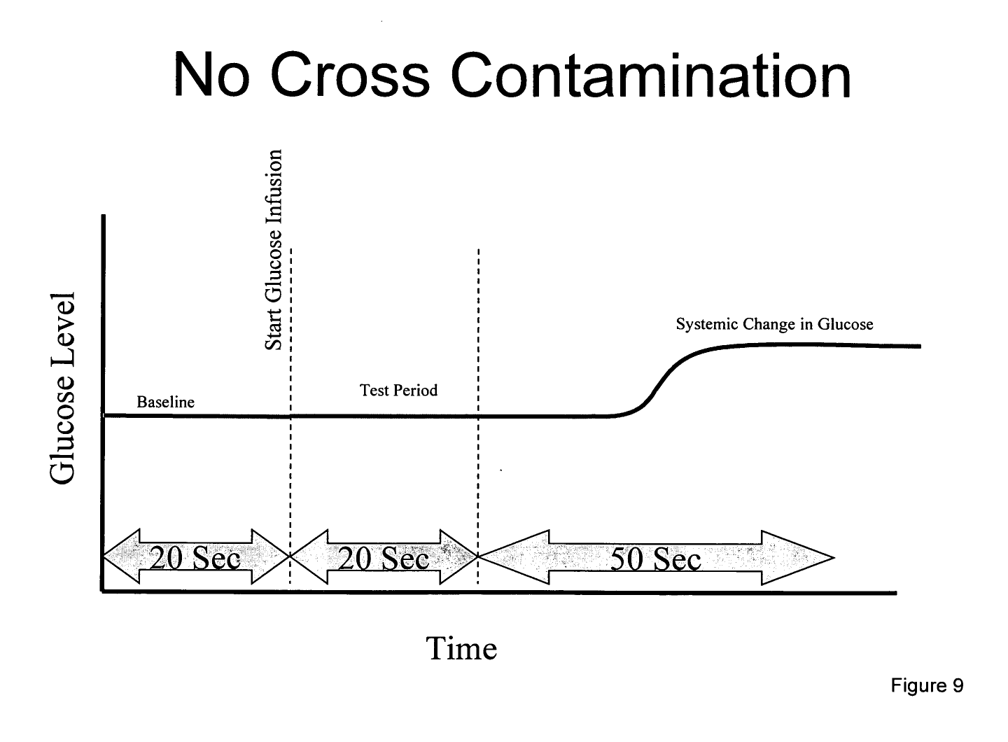

Animal testing. A cross-contamination study on a mechanically ventilated pig was conducted to complete the investigation into cross-contamination. The protocol for investigation was (1) Place the catheter and confirm location by fluoroscopy; (2) Evaluate flow characteristics by injecting contrast agent; (3) Evaluate for cross-contamination; (4) Move catheter to next location. The testing procedure was
1. Initiate a sampling period where blood samples are acquired from the catheter every 4 seconds, as in FIG. 7. The actual circuit used for the test is shown in FIG. 8.
2. The initial phase establishes a baseline glucose level, as shown in FIG. 9.
3. Initiate an infusion of 50% glucose at a rate of 1000 ml/hr for a duration of 20 seconds, as shown at the start of infusion in FIG. 9.
4. Continue acquiring samples for the duration of the infusion and for a period of 50 seconds after infusion stopped.
5. Measure the glucose levels in the samples obtained.

Evaluation of Results. In a condition without cross-contamination, the initial glucose levels and those during glucose infusion will be approximately equivalent until the infused glucose has circulated in the vascular system. The amount of infused glucose will result in approximately a 50 mg/dl systemic change assuming a total blood volume of 5 liters. This end of study glucose level will be referred to as the ending glucose level. FIG. 9 is an illustration of an idealized response when no cross-contamination is present.

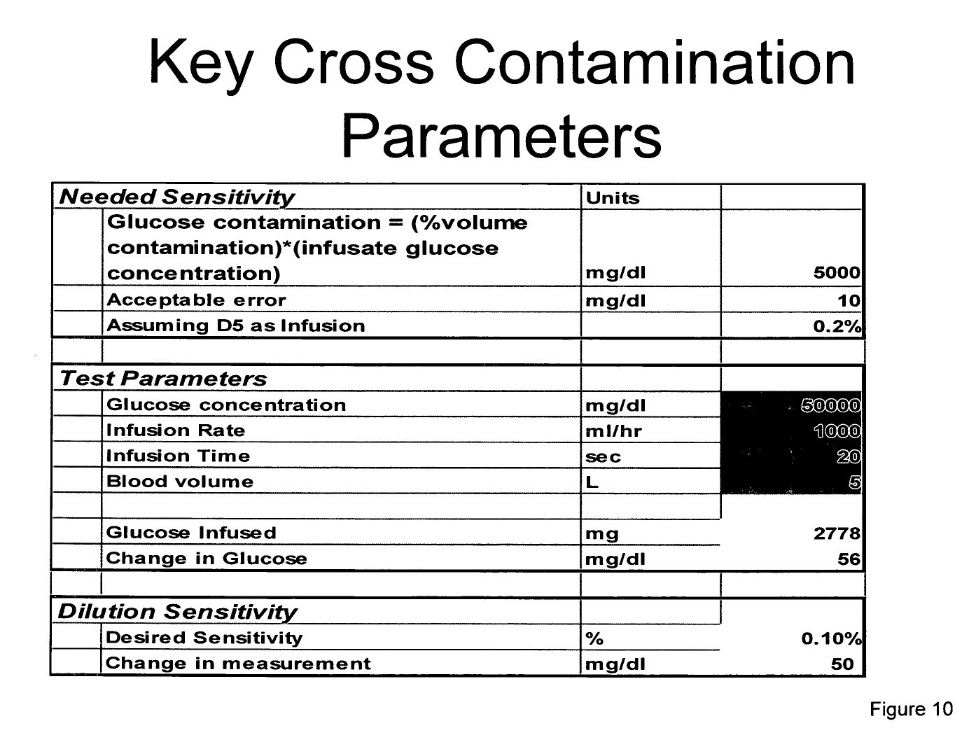

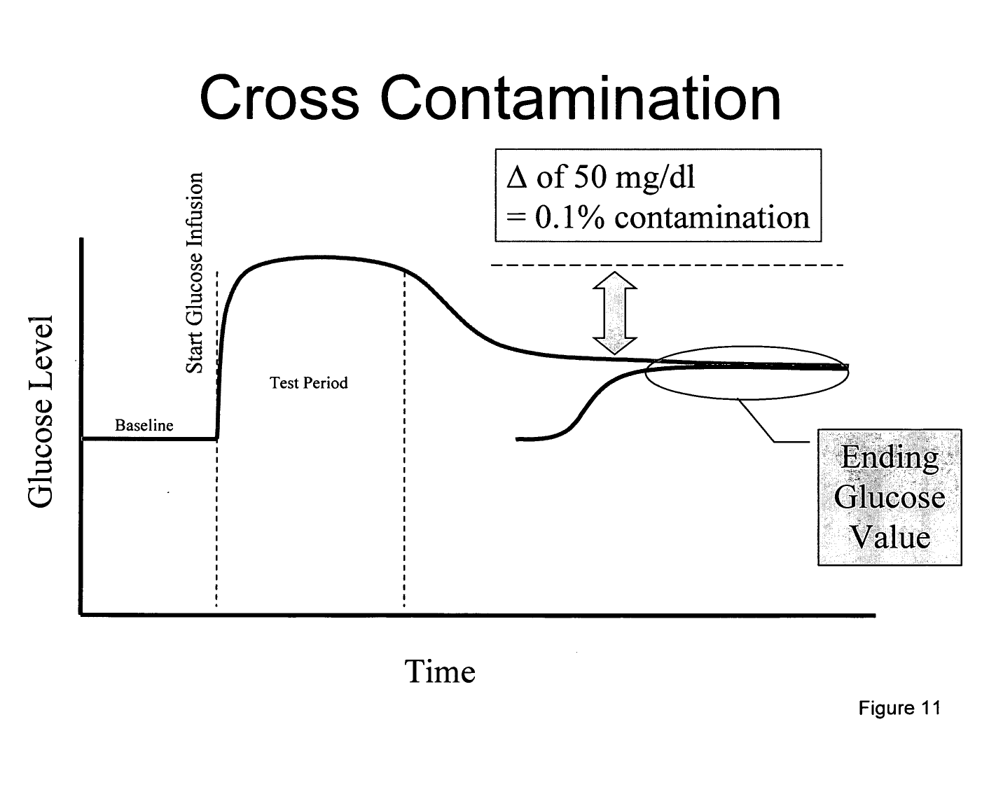

If cross-contamination occurs as a result of the infused glucose then the measured glucose will increase concurrently with the start of the glucose infusion. The use of a 50% glucose solution results in a significant glucose change even when the percentage of cross-contamination is less than 1%. FIG. 10 provides a reasonable outline of the key study parameters. If the acceptable error of cross-contamination is defined as 10 mg/dl and the solution being infused is 5% glucose, then the maximum acceptable percentage of contamination is 0.2%. If cross-contamination does occur during the glucose infusion stage, the amount of change can be easily detected. By using a 50% glucose solution (50,000 mg/dl) a 0.1% cross-contamination will result in a 50 mg/dl change relative the end of study glucose level. As shown in FIG. 11, cross-contamination results in a rapid rise during infusion with a decrease to the end of study glucose level. The maximum measured glucose level is then compared to the end of study glucose level (indicative of the final systemic glucose level) and a simple subtraction performed. A 50 mg/dl increase is indicative of approximately 0.1% cross-contamination while 100 mg/dl is indicative of 0.2% cross-contamination. In the clinical setting where 5% glucose solutions are commonly used 0.2% cross-contamination would result in glucose over prediction of 10 mg/dl.

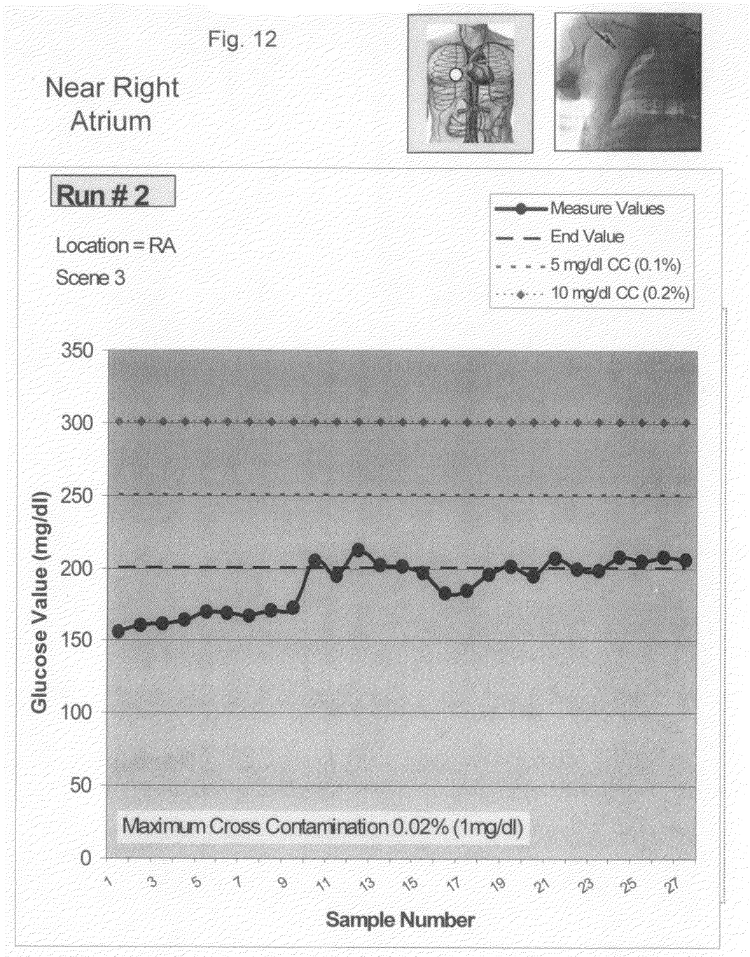

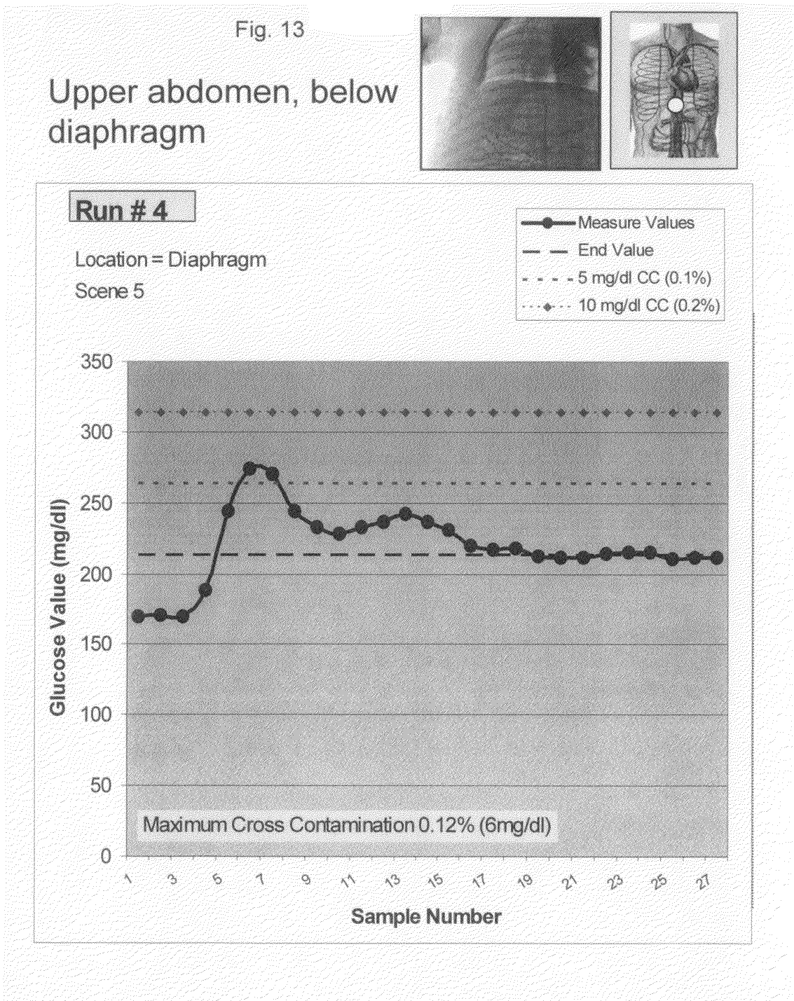

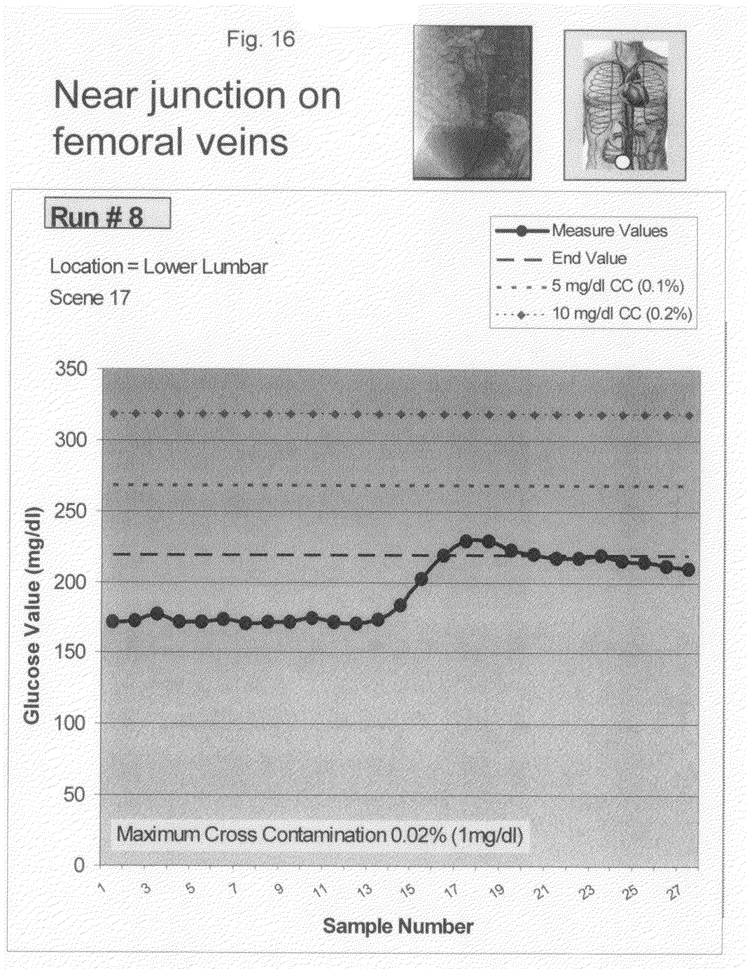

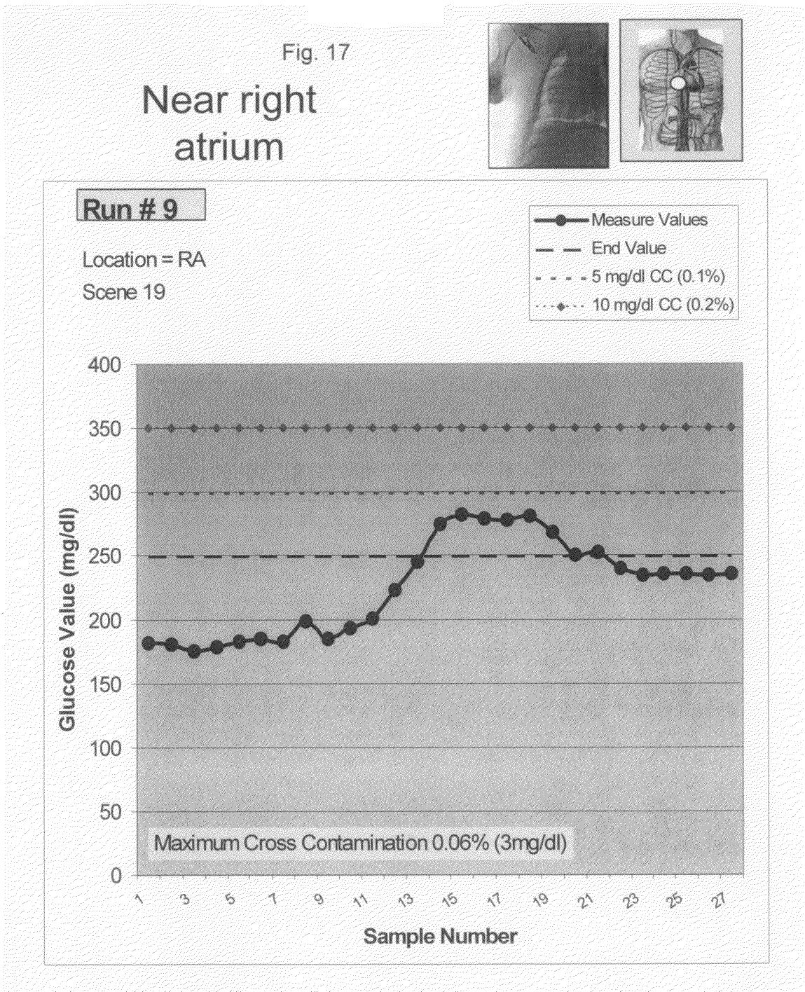

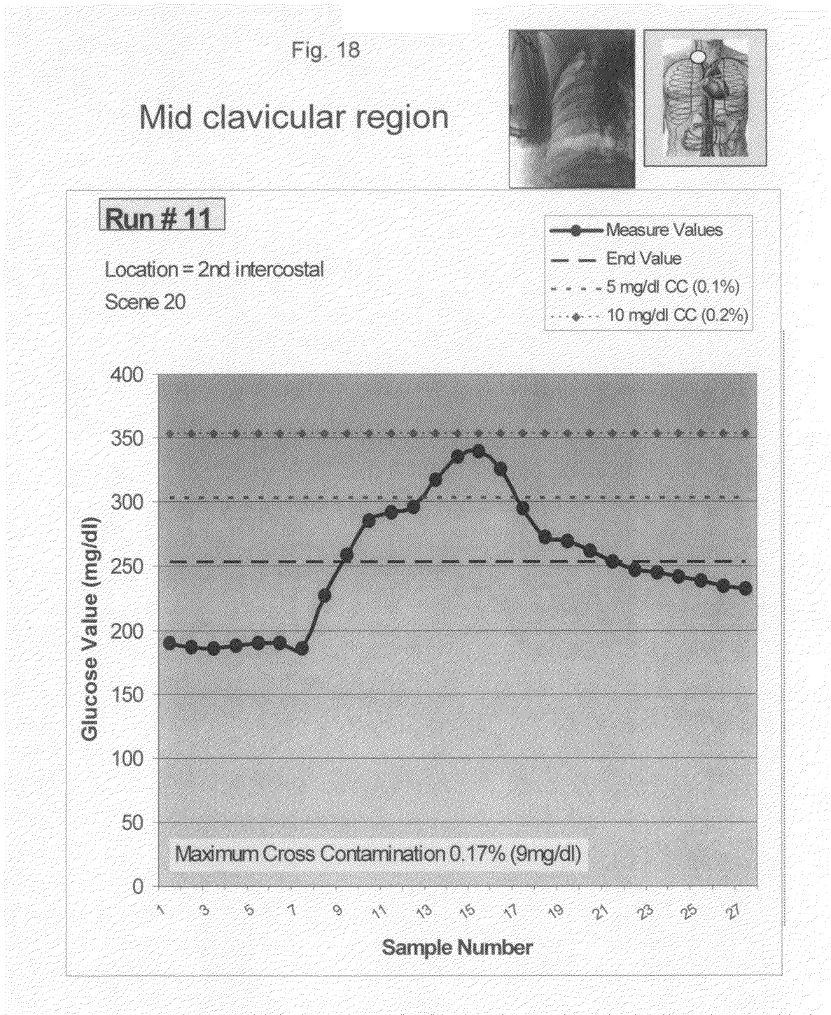

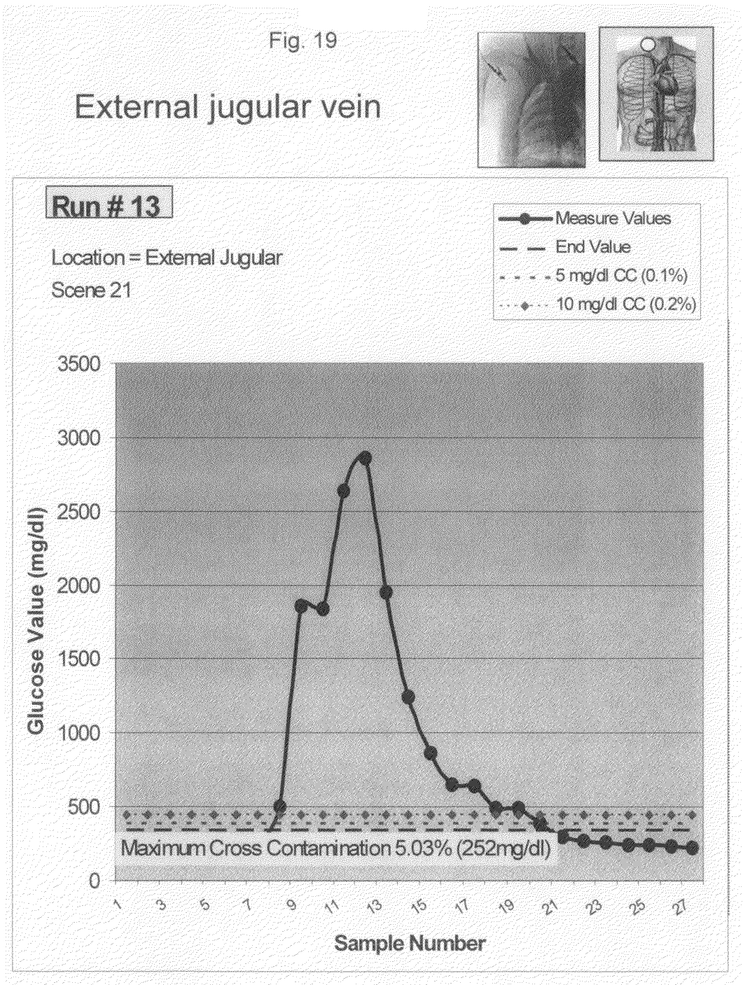

FIG. 12-19 are illustrations of experimental results, summarized in Table 4. In each figure, the radiographic image on the left side indicates catheter location. The vascular diagram shows the catheter location relative to the overall vasculature system. The graph shows test results. The x-axis is the sample number procured over the approximately 2 minutes of testing. The y-axis is the measured glucose concentration. The lowest horizontal line is the end of study glucose value which corresponds to the systemic increase in glucose concentration due to the glucose infusion. The next line is 50 mg/dl higher and corresponds to 0.1% contamination. The next line is 100 mg/dl higher then the end of study line and corresponds to 0.2% contamination. The glucose measurements from the study are plotted on the same axis.

TABLE 4

| | | | % Cross- |
|---|---|---|---|
| Catheter Location | Figure Number | Ventilation | contamination |
| Near right atrium | 12 | Yes | 0.02% |
| Upper abdomen | 13 | Yes | 0.12% |
| Mid abdomen | 14 | Yes | 0.26% |
| Mid Abdomen | 15 | NO | 0.06% |
| Junction of femoral veins | 16 | Yes | 0.02% |
| Right atrium | 17 | Yes | 0.06% |
| Mid clavicular | 18 | Yes | 0.17% |
| External jugular | 19 | Yes | 5.3% |

Four of the seven locations resulted in cross-contamination greater than 0.1%. This contrasts with the results anticipated and obtained from the computational fluid dynamics study and the laboratory investigation.

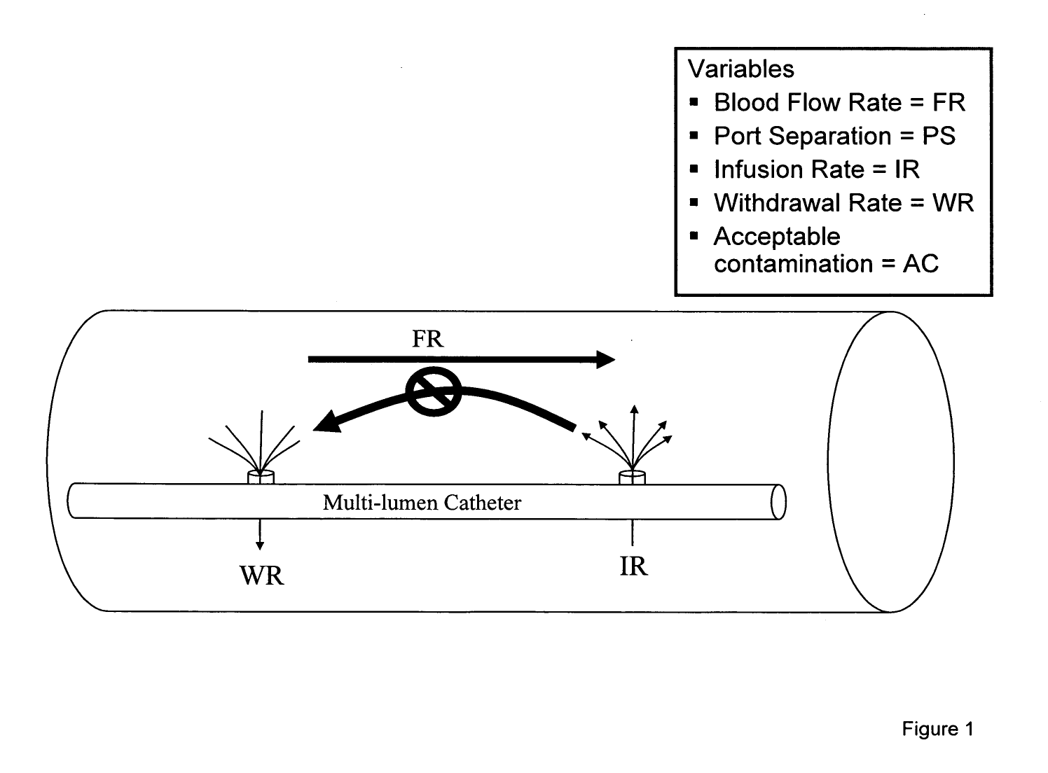

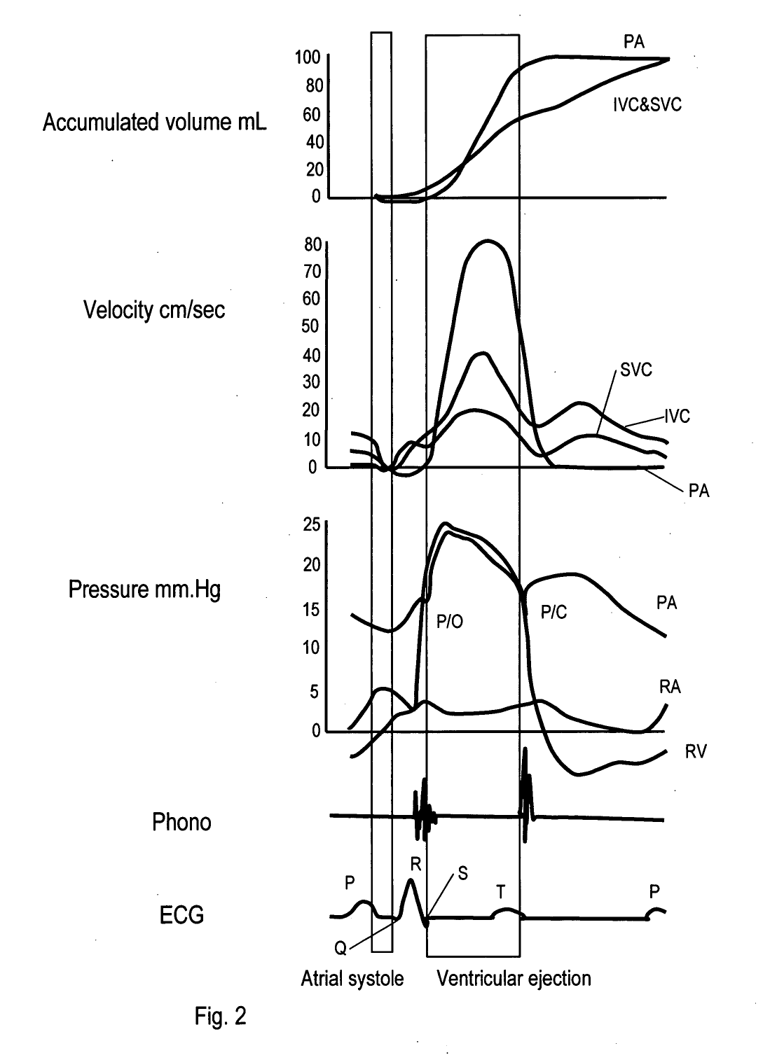

Mechanism for cross-contamination. As discussed in conjunction with FIG. 1, conditions of stagnant flow or reversed flow from the distal end of the catheter to the proximal end can result in cross-contamination. Any medical state, physiological condition or medical treatment of the subject that results in retrograde flow in large venous vessels creates an opportunity for cross-contamination. A number of medical conditions or treatments can cause such a retrograde flow; two common causes of retrograde flow in the vena cava are mechanical ventilation and abnormal cardiac function.

The normal venous pulse (Jugular venous pulse, JVP) reflects phasic pressure changes in the right atrium and consists of three positive waves and two negative troughs. In considering this pulse it is useful to refer to the events of the cardiac cycle. The positive presystolic “a” wave is produced by right atrial contraction and is the dominant wave in the JVP particularly during inspiration. During atrial relaxation, the venous pulse descends from the summit of the “a” way. Depending on the PR interval, this descent may continue until a plateau (“z” point) is reached just prior to right ventricular systole. More often the descent is interrupted by a second positive venous wave, “c” wave, which is produced by a bulging of the tricuspid valve into the right atrium during right ventricular isovolumic systole and by the impact of the crowded artery adjacent to the jugular vein. Following the summit of the “c” wave, the JVP contour declines, forming the normal negative systolic wave, the “x” wave. The “x” descent is due to a combination of atrial relaxation, the downward displacement of the tricuspid valve during right ventricular systole, and the ejection of blood from both the ventricles.

In the case of abnormal cardiac function, at least three mechanisms are known to cause a retrograde flow: tricuspid valve regurgitation, increased flow resistance out of the right atrium, and atrial fibrillation. In the case of tricuspid regurgitation, the right ventricle contracts but the tricuspid valve does not prevent retrograde flow into the right atrium and subsequently the thoracic veins. Possible conditions of retrograde flow can be associated with larger than normal “a” waves. Giant “a” waves are present with each beat, the right atrium is contracting against an increased resistance. This may result from obstruction at the tricuspid valve (tricuspid stenosis or atresia), right atrial myxoma or conditions associated with increased resistance to right ventricular filling. Abnormally large “a” waves can occur in patients with pulmonary stenosis or pulmonary hypertension in whom both the atrial and right ventricular septa are intact. Abnormally large and typically narrow “a” waves, often referred to as Cannon “a” waves, occur when the right atrium contracts while the tricuspid valve is closed during right ventricular systole. Cannon waves can occur either regularly or irregularly and are most common in the presence of arrhythmias. Atrial fibrillation is a condition known to cause the irregular occurrence of cannon “a” waves.

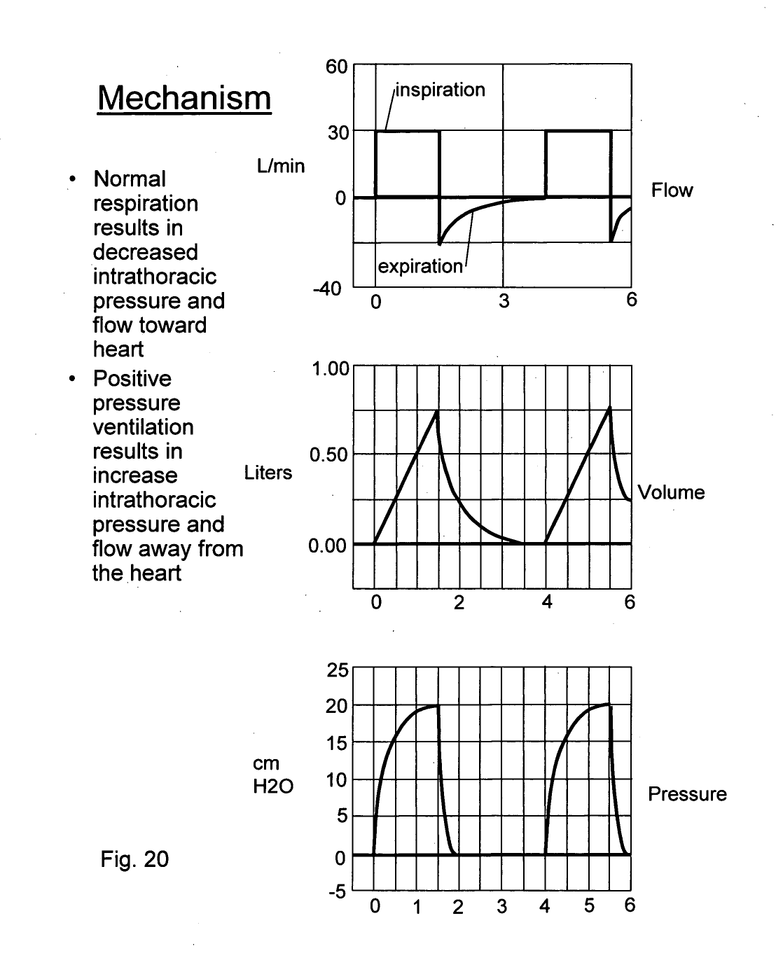

Another known source of stagnant or retrograde flow is mechanical ventilation. During normal breathing the diaphragm is lowered creating a negative pressure in the thoracic cavity. This negative pressure creates the gradient for air movement and for the filling of the lungs with each new breath. The negative pressure in the thoracic cavity also helps blood return to the heart. In the case of positive pressure ventilation, the pressure gradients are reversed. As shown in FIG. 20, the process of inflating the lung results in increased thoracic pressures. The impact of positive pressure ventilation on right heart filling pressures and volume has been documented in the literature. See, e.g., Principles and Practice of Mechanical Ventilation, by Martin J. Tobin, McGraw-Hill, copyright 2006, incorporated herein by reference. Additionally other peer-reviewed publications review the interactions between positive pressure ventilation and heart function. See, e.g., “Heart-lung interactions: applications in the critically ill” by H. E. Fessler, European Respiratory Journal, 1997; 10: 226-237, and “Cardiovascular Issues in Respiratory Care” by Michael R. Pinsky, Chest 2005: 128: 592-597; each of which is incorporated herein by reference. The impact on blood flow in the large veins leading to the heart was investigated in the 1960s but has received very little documentation or re-examination since then. Key papers covering blood flow in the large thoracic vessels are as follows and are incorporated herein by reference: Chevalier P A, Weber K C, Engle J C, et al. Direct measurement of right and left heart outputs in ValSalva-like maneuver in dogs. Proc Soc Exper Biol Med 1972; 139:1429-1437.: Guntheroth W C, Gould R, Butler J, et al. Pulsatile flow in pulmonary artery, capillary and vein in the dog. Cardiovascular Res 1974; 8:330-337.: Guntheroth W G, Morgan B C, Mullins G L. Effect of respiration on venous return and stroke volume in cardiac tamponade. Mechanism of pulsus paradoxus. Circ Res 1967; 20:381-390; Holt J P. The effect of positive and negative intrathoracic pressure on cardiac output and venous return in the dog. Am J Physiol 1944; 142:594-603; Morgan B C, Abel F L, Mullins G L, et al. Flow patterns in cavae, pulmonary artery, pulmonary vein and aorta in intact dogs. Am J Physiol 1966; 210; 903-909; Morgan B C, Martin W E, Hornbein T F, et al. Hemodynamic effects of intermittent positive pressure respiration. Anesthesiology 1960; 27:584-590. Upon review of the above literature, there are a number of unobvious characteristics of the large veins that enable mechanical ventilation induced retrograde flow. First, the superior and inferior vena cava do not have valves that prevent reverse flow. In the smaller veins of the body there are one way valves that allow flow toward the heart but not retrograde flow. The lack of valves in the vena cava creates an opportunity where blood can flow toward the heart or away from heart solely based upon pressure. Additionally this compliant effectively runs across three different atmospherically related but different segments. The segments for examination are the abdominal cavity, the thoracic cavity and the ambient/jugular cavity. Large asymmetric pressure changes in any of these segments can induce flow within the vena cava.

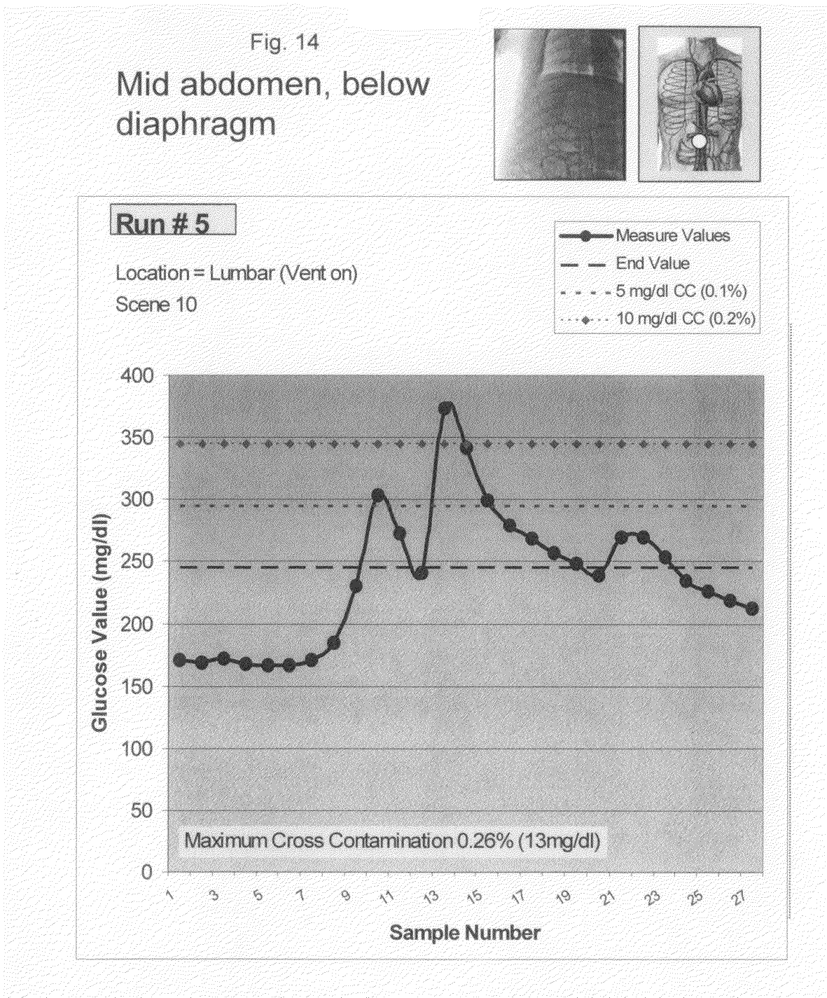

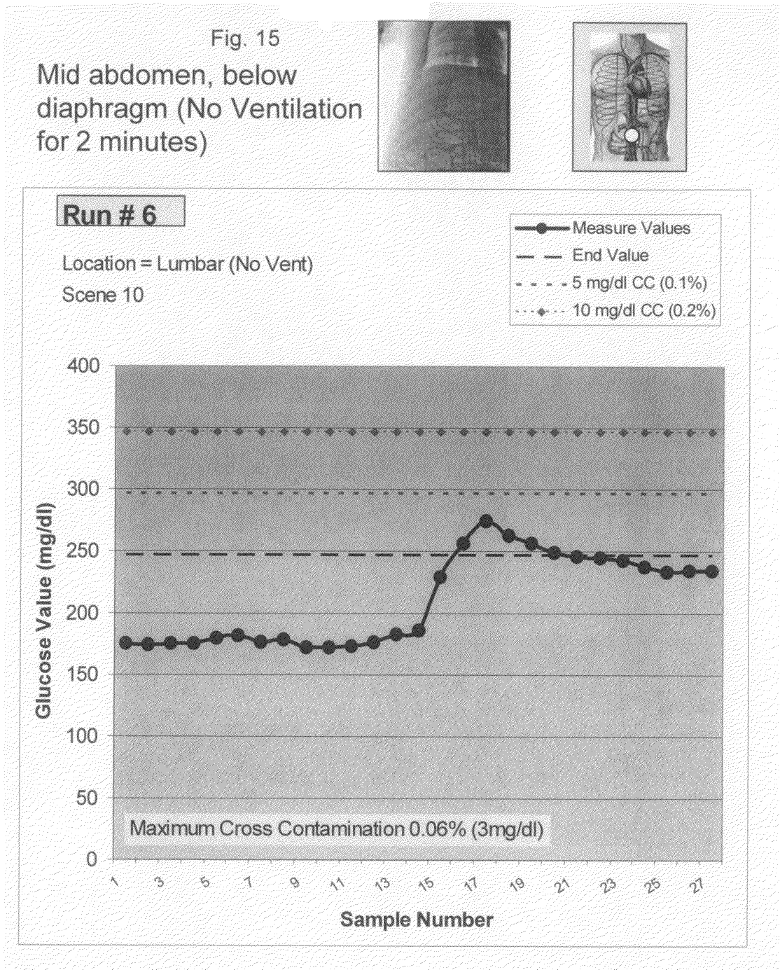

In the study animal conducted, reverse flow occurred during the periods of positive pressure ventilation. To help confirm that mechanical ventilation is the major source of retrograde flow and subsequent contamination, one location was examined with and without ventilation. For the catheter location in the mid abdomen, two tests were conducted. The first was conducted with mechanical ventilation on and the second test with no ventilation. The rate of ventilation was 10 breaths per minute. As can be seen by comparing FIG. 14 and FIG. 15, the degree of cross-contamination is very significant when the animal was ventilated while there is little or no evidence of contamination when the ventilation was stopped for the duration of the study. Careful examination of FIG. 14 also shows a variation of cross-contamination that has a frequency that is well correlated with the ventilation frequency. Since the pressure gradients vary over the ventilation cycle, the amount of cross-contamination can vary as a function of these changes.

Detection of cross-contamination. Reliable detection of conditions that are likely to lead to cross-contamination can be beneficial, since glucose measurements made during such conditions can be adjusted or discarded as possibly inaccurate. Pressure changes can be used to detect conditions likely to lead to cross-contamination. One mechanism that creates retrograde blood flow resulting in contamination of the blood sample is a change in the pressure surrounding the vessel. An increase in pressure can result in compression of the vessel and flow away from the compression. Pressure changes can occur within the thoracic cavity, and can occur within the abdominal cavity. Pressure can be sensed within the venous system. If the pressure change is significant enough to be likely to cause cross-contamination, then the system can indicate the potential for cross-contamination to allow appropriate actions to be taken (e.g., no sample withdrawal, disregard measurements on this sample that are susceptible to the cross-contaminating substance(s), disregard this sample, etc.). Abnormal cardiac function resulting in retrograde flood flow is commonly associated with abnormally large “a” waves. This degree of pressure variation can be sensed by an intravascular pressure monitoring system. A pressure sensing system for these types of waves can be based upon both the magnitude and/or the frequency of occurrence. In addition to pressure changes due to mechanical ventilation, the following are known to change pressure changes which can influence venous flow: valsalva maneuver, abnormal cardiac function, coughing or gross movement of the body. A valsalva maneuver is performed by forcibly exhaling against closed lips and pinched nose or is frequently used when defecating.

Many types of abnormal cardiac function, specifically atrial fibrillation, are associated with an abnormal electrocardiogram, EKG. An additional level of protection against potential cross-contamination due to abnormal cardiac function can be incorporated by ensuring that the heart has a normal EKG.

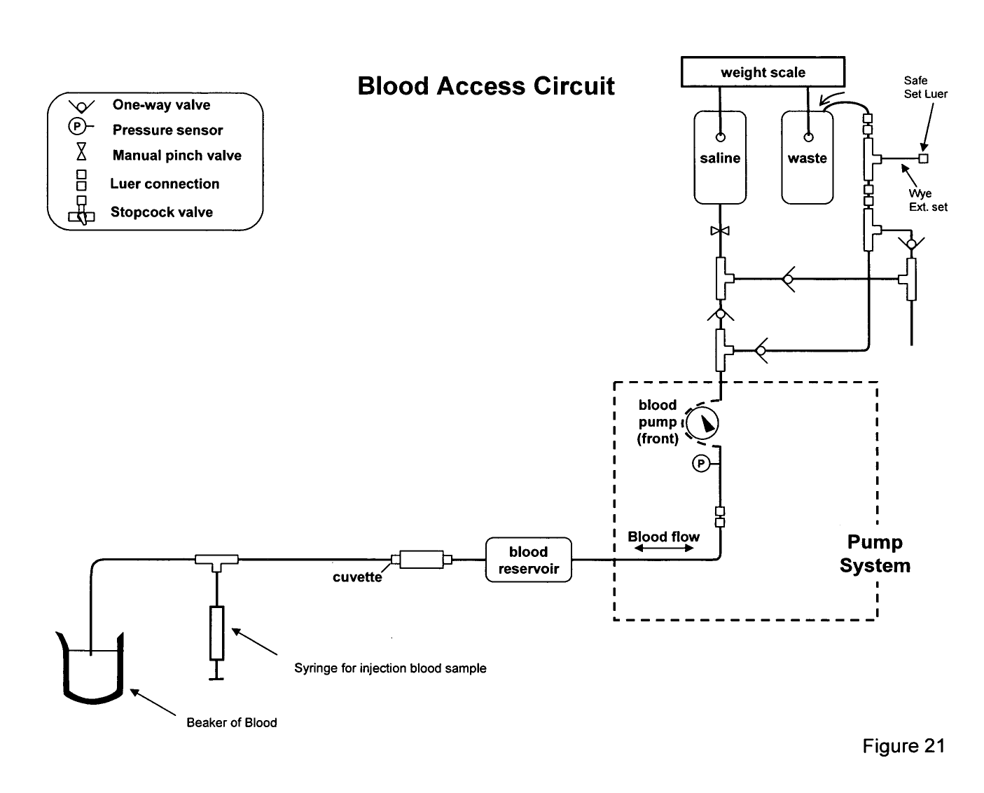

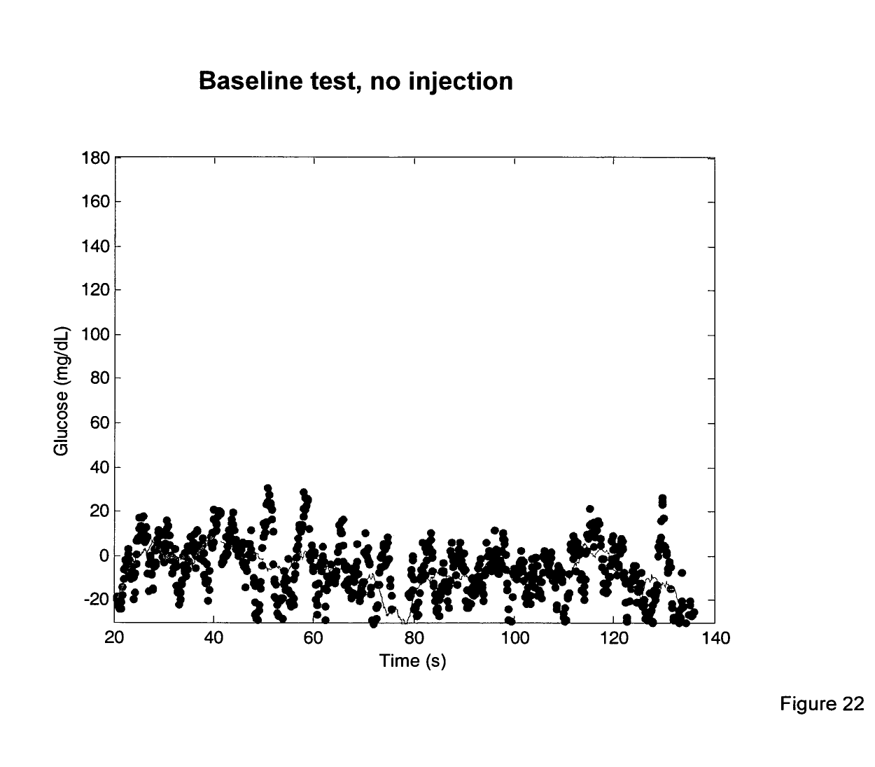

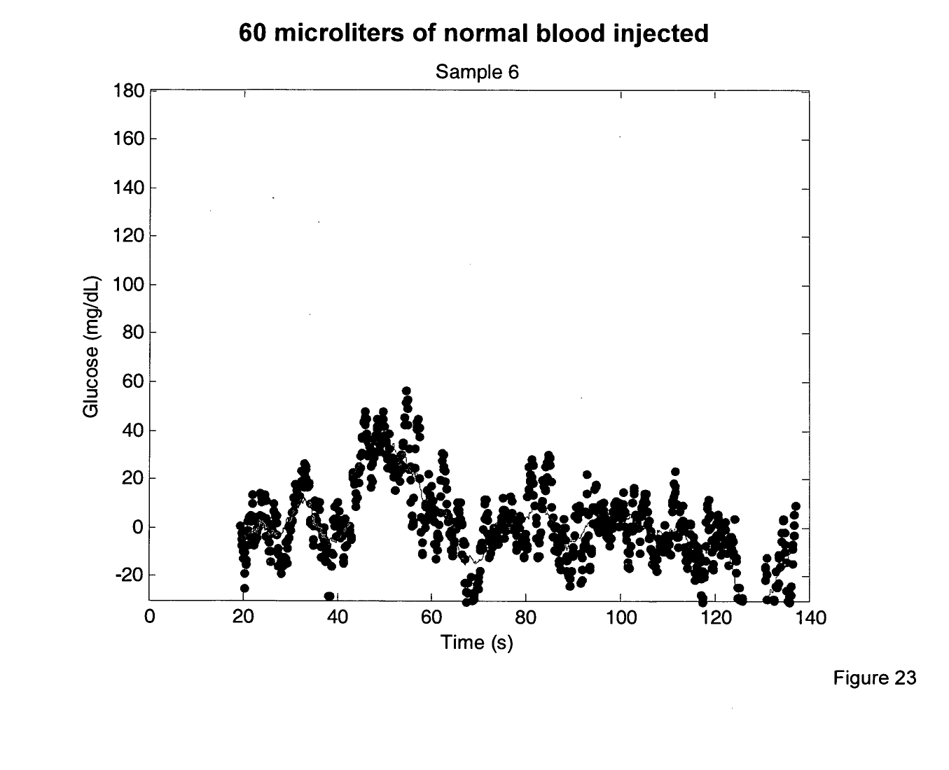

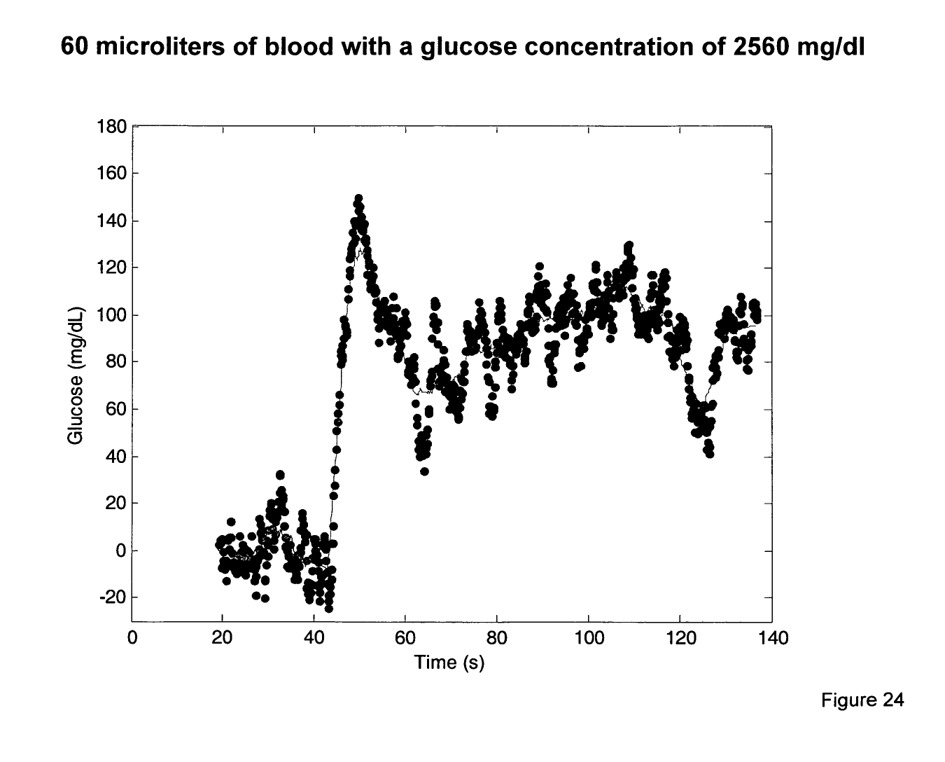

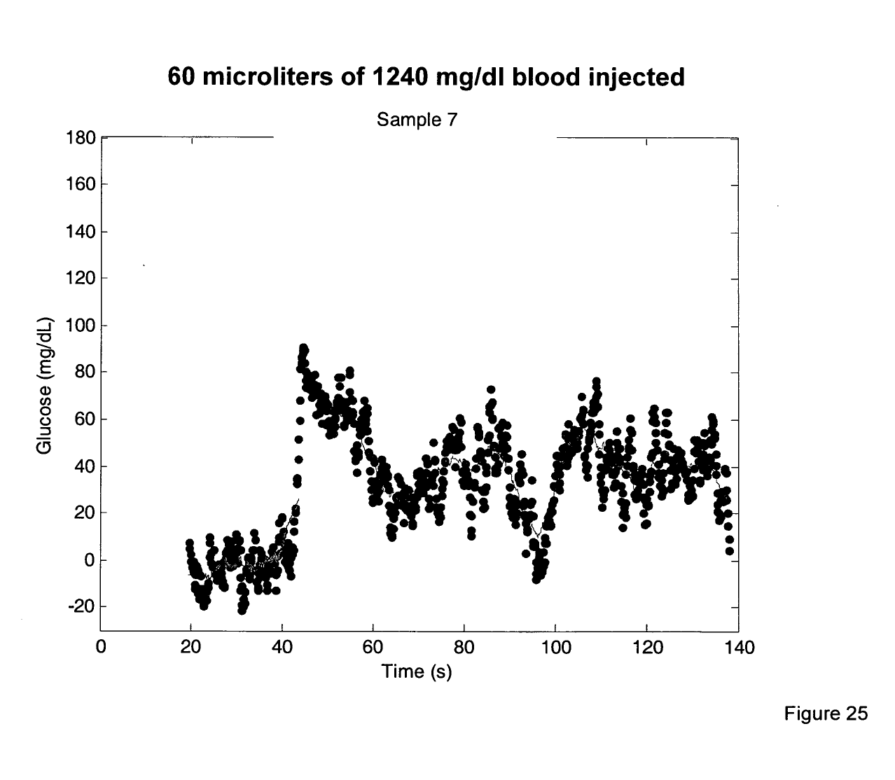

The measured glucose values can themselves be used to detect when one or more measured values are likely to have been compromised by cross-contamination. Cross-contamination due to mechanical ventilation has a frequency that is correlated with the ventilation rate. Maximum cross-contamination can occur during the highest rates of pressure change and then decrease. If continuous, or suitably frequent, measurements are made, these measurements will have a variation in glucose values that have a frequency that mimics that of the ventilator. A system can make continuous or suitably frequent measurements that span at least one ventilation cycle and check for significant variation in the glucose values. Typical physiology results in glucose variations that follow general trends and have a maximum rate of about 2 mg/dl per minute. If the measurement was to show sinusoidal variations, or other variations correlated with ventilator operation and not with physiological activity, such variations would be highly indicative of cross-contamination. The system can automatically assess the stability of the glucose measurement and provide a warning indication the possible presence of cross-contamination. To demonstrate the influence of cross-contamination a study was conducted. FIG. 21 shows the blood access circuit used for demonstration of measurement instability due to cross-contamination. Blood is a drawn into the circuit in a standard manner but a 60 microliter bolus of blood is inserted at the T-junction during the withdrawal process. In actual practice there is a very short pausing of the withdrawal pump to enable the infusion to be made. The bolus of blood can contain a glucose concentration that is the same as that of the beaker or can mimic a concentration level that could be observed during cross-contamination. In the testing conducted two different glucose levels were examined, 1240 mg/dl and 2560 mg/dl. FIG. 22 shows the overall stability of the measurement during the withdrawal period when the system is simply pulling blood from the beaker. The individual points depicted on the graph are optical glucose measurements made every one second while the solid line represents an average of eight seconds. As can be seen in this figure the overall stability of the glucose measurement is quite good and varies by approximately ±20 mg/dl. FIG. 23 is a control figure showing the influence of injecting the 60 microliter bolus but where the blood bolus has the same glucose concentration as the blood being withdrawn from the beaker. There is some minor decrease in the stability of the measurement at approximately 40 seconds due to turning on and off the pump. FIG. 24 shows the impact of a 2560 mg/dl bolus. In addition to the rapid rise from the baseline glucose measurements there is a residual “ringing” or instability due to the fact that some of the high concentration blood does not exit the optical measurement location. FIG. 25 shows the impact of a 1240 mg/dl bolus. As was previously observed there is a large initial increase from the baseline glucose measurement as well as a residual instability. As evidenced by these figures, any contaminated blood drawn into the circuit will result in a measurement instability that can be detected.

The physics describing the potential for cross-contamination indicate that the amount of cross-contamination can be sensitive to the withdrawal rate. In the case of constant glucose concentration in a large volume of blood, the amount of glucose in the sample is not dependent upon the rate of withdrawal. In contrast, when cross-contamination occurs, the rate of withdrawal influences the amount of contaminated sample obtained. As blood is removed from the body and mixing occurs in the withdrawal system, higher withdrawal rates lead to more contaminated sample. Additionally a high withdrawal rate can pull downstream blood back into the catheter increasing the amount of contamination. The relationship between withdrawal rate and the amount of cross-contamination can be used to identify situations where cross-contamination occurs. The measurement system can make measurements at two or more different withdrawal rates. A difference between the measured glucose values can be an indicator of contamination. In the case of a pressure limited withdrawal (i.e., where withdrawal rate is variable to maintain a substantially constant withdrawal pressure), the rate of flow varies over the withdrawal period due to the presence of blood in the system. Glucose consistency over the withdrawal period will be at the measurement or noise level of the system if no contamination is present. A change in measured glucose larger than the typical maximum physiologic rate of 2 mg/dl per minute can indicate that cross-contamination has occurred.

Cross-contamination can be detected by comparing two different analyte values. A first analyte value determined can be an analyte that undergoes volumetric dilution. For example cross-contamination will result in a decreased hematocrit value due to volumetric dilution. A second value determined can be an analyte that is present in the infused fluid. For example cross-contamination by a glucose-containing infusate will result in an increased glucose measurement value but will concurrently result in a decreased measurement of an analyte not contained in the infused solution (due to volumetric dilution of the sample by the infusate). The method of analyte comparison can be conducted under any situation what would vary the amount of contamination, for example measurement at different conditions in the ventilator cycle or measurements at different withdrawal rates. In case of varying the withdrawal rate the system can be operated as follows (1) measure both hematocrit and glucose at a high withdrawal rate, (2) measure both hematocrit and glucose at a lower withdrawal rate and (3) compare the results. If no cross-contamination is occurring there should be substantially no alteration in the hematocrit to glucose ratio. If cross-contamination is occurring the hematocrit value will increase as the withdrawal rate is decreased and the glucose value will decrease.

Cross-contamination can be assessed by making two measurements where the difference between the measurements is the operation of the infusion pumps. The method for assessing cross-contamination based upon one or more measured results has been described above. The difference in this methodology is the fact that the withdrawal rate can be maintained at a constant level but the conditions under which the two withdrawals are made are different. In addition to comparing specific blood analyte values, a general spectroscopic comparison can be utilized. The spectral response of the blood sample is the aggregate influence of all of the absorbing compounds in the sample. Therefore if the composition of the sample has changed appreciably a comparison between two spectroscopic responses taken under two different conditions can be utilized to identify contamination. For example, if sample #1 is contamination free while sample #2 contains the contaminant a simple spectral comparison by subtraction or division or a related method will show a spectroscopic response above the baseline noise level. Such an observation can be highly indicative of the presence of cross-contamination. Therefore a general spectroscopic similarity metric such as a ratio can be utilized to determine the presence of cross-contamination. The above process can be repeated, e.g., every time a new infusion is started for a given patient.

Reducing the influence of cross-contamination. It can be convenient for a measurement system to automatically adjust its operation to reduce the influence of cross-contamination. As one example, if the measured response shows variations in glucose values that are consistent with the ventilation frequency then the resulting data stream can be processed to remove the values likely to be influenced by cross-contamination. In a simple example, the lowest 10% of values in a sequence of measurement values can be averaged and this number be reported as the measured glucose value. More sophisticated process methods such as digital filtering or Fourier transformation can also be used.

Since cross-contamination results from pressure changes, the change in pressure can be recorded and measured glucose values adjusted based on the pressure changes. For example, if the pressure changes exceeded a threshold then measurements made on blood procured during this period can be excluded from the analysis. In another example, the pressure change can be used as a correction for the measured result. For example the measured glucose values can be adjusted based on a function of the pressure changes, e.g., a fixed percentage, a percentage determined based on the magnitude of the pressure difference, or a percentage determined based on the pressure differences and the composition of the infusate. The adjustment to be made can also be tailored to a specific patient by combination with other methods—e.g., the pressure difference can be recorded, and the actual glucose value at that pressure difference determined by another correction method (e.g., interpolation between values obtained under conditions of no cross-contamination), and the difference between the measured value and the corrected value used to determine the correction to be applied to subsequent measurements under similar conditions such as pressure differences.

During the standard ventilation cycle the flow rate in the vena cava varies. The periods of decreased or reversed flow represent the greatest potential for cross-contamination during the ventilation cycle. If there is potential for cross-contamination to occur during these periods of decreased or reversed flow in the blood vessel, the problem can be mitigated by stopping the withdrawal during these periods. A variable rate withdrawal can be linked to the ventilation cycle by monitoring the pressure in the vessel or by monitoring the pressure of the ventilator airway. The withdrawal rate can be accordingly modulated such that the withdrawal rate is decreased appropriately during periods when cross-contamination is likely to be present. The modulation process can involve decreasing the withdrawal rate, stopping the withdrawal or even reversing the withdrawal rate to insure that the sample obtained is free from cross-contamination.

In some blood access system there is the opportunity to withdraw blood from the central venous catheter and to physically separate potentially cross-contaminated blood from uncontaminated blood. In one example, a pressure sensor can identify a pressure change indicative of a flow reversal. The blood segment associated with this potentially cross-contaminated sample can be tracked as it is withdrawn through the blood access system. At an appropriate point the potentially contaminated sample can be diverted into a second fluid path such that the resulting blood sample for measurement is contamination free.

Infusions that can result in cross-contamination can also be stopped during phases of the ventilator cycle likely to lead to cross-contamination. The trigger for stopping fluid infusion can be an intra vessel pressure or flow measurement, a direct trigger signal from the ventilator, measured flow or pressure from the ventilator patient circuit, or some other method that provides information regarding the status of ventilation including nerve impulse or EMG signals, intra-abdominal, intrathoracic or intravascular pressure signals.

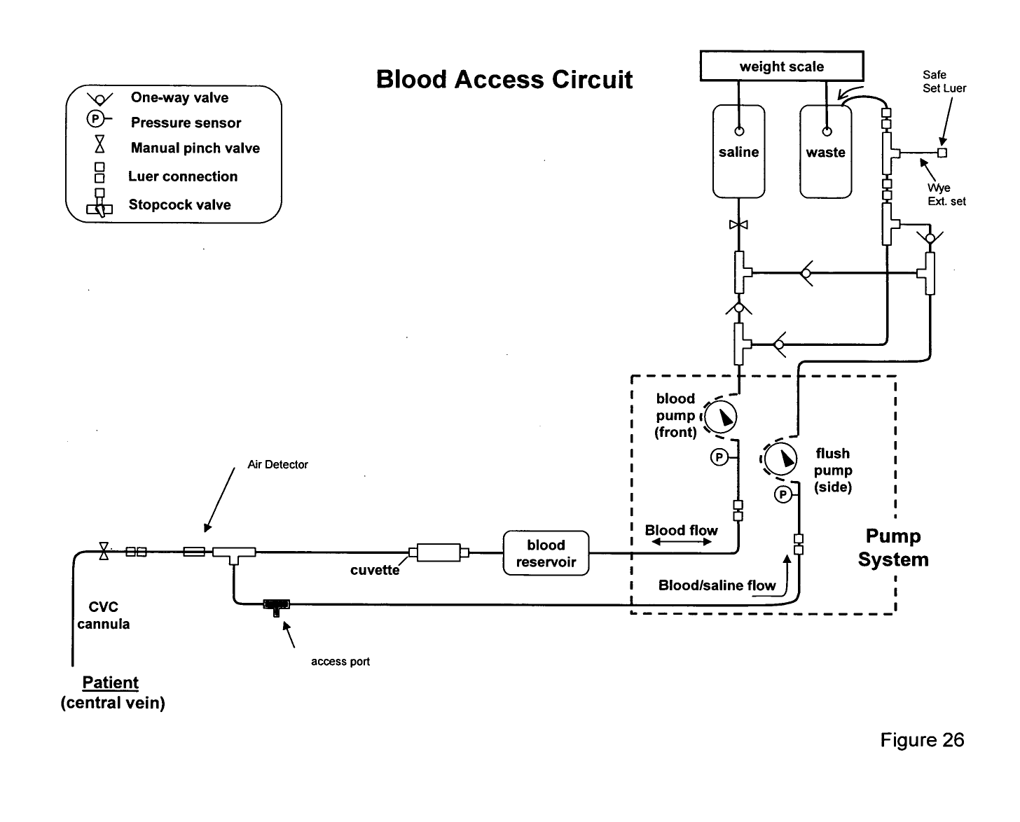

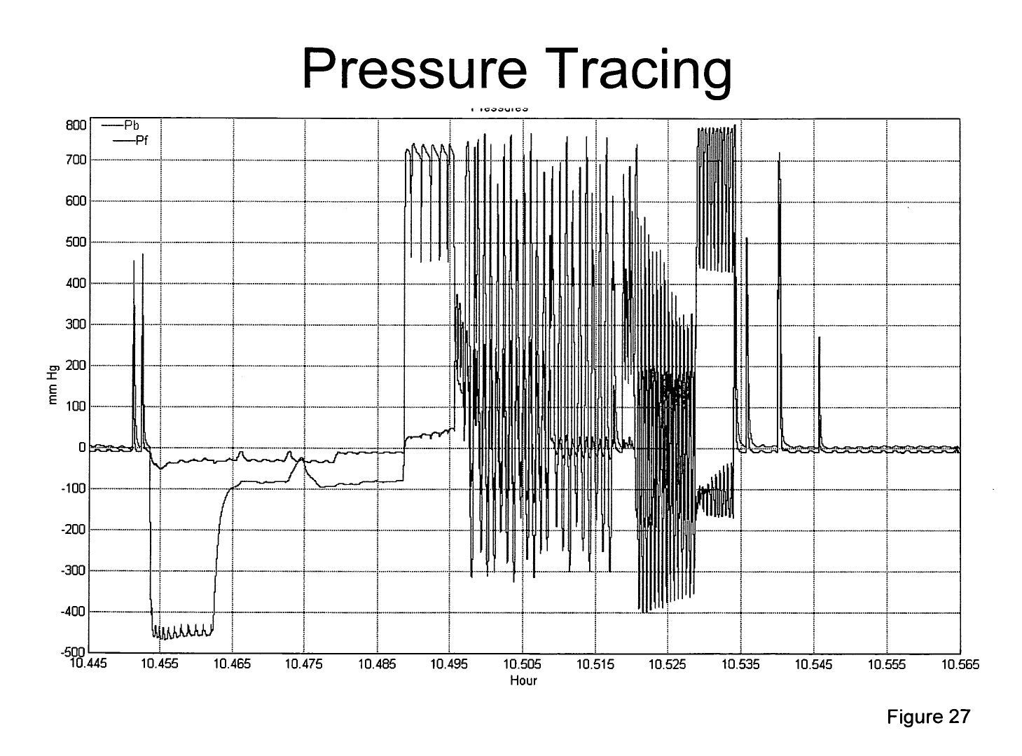

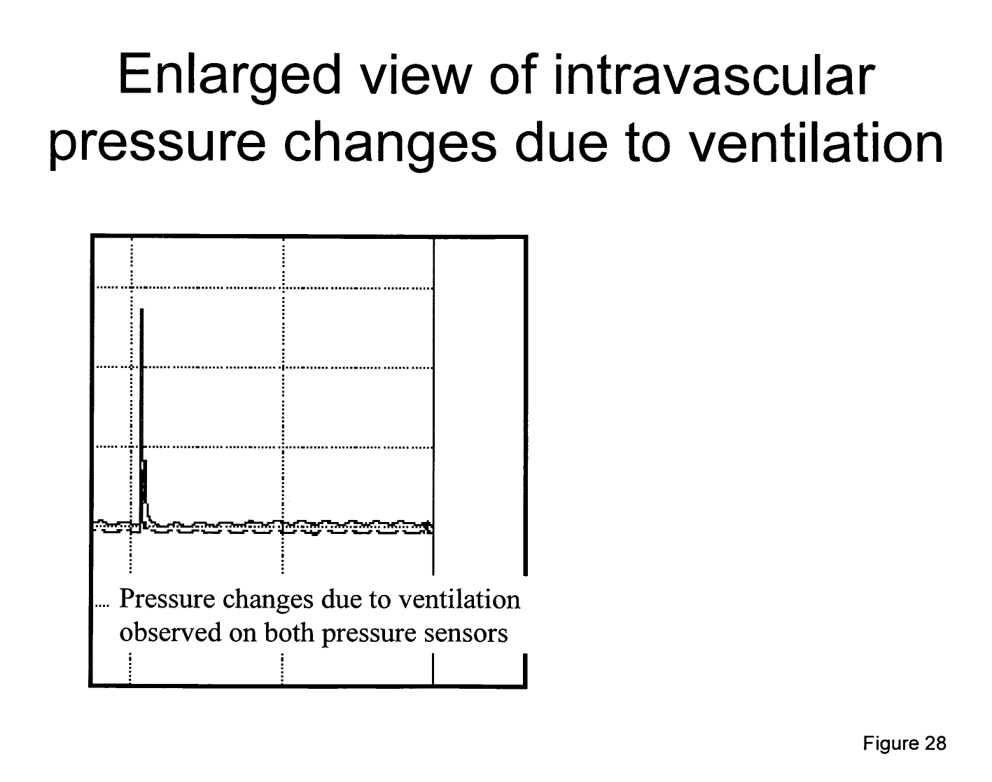

Under conditions where the patient is not ventilated or the influence of ventilation is moderately small, the withdrawal rate can be a more important factor. In the animal testing conducted with catheter locations near the right atrium or in the pelvis, there was no appreciable cross contamination but the withdrawal rate was only 20 ml/min. A nurse can easily generate withdrawal rates in excess of 60 ml/min. The potential for cross-contamination is influenced by the flow rate of blood at the site of the central venous catheter, the rate of infusion, the rate of withdrawal, the glucose concentration of the infused fluid, catheter port orientation and the distance between the point of infusion and withdrawal. The rate of withdrawal is an important parameter in determining cross-contamination: control of this parameter can reduce the likelihood of cross-contamination. In the hospital environment the rate of withdrawal can vary appreciably due to the type of syringe used, the force applied by the nurse or clinical care provider, and a variety of other uncontrolled variables. Under a variety of conditions, the withdrawal rate of the blood access system can specified and controlled such that the amount of cross-contamination does not affect the clinical efficacy of the device. Based upon medical data, the typical flow in a non-ventilated patient in the superior vena cava will average between 10 and 20 cm per second with a peak at 35 cm per second in the direction towards the heart. This will overwhelm both the infusion velocity and the withdrawal velocity of the infused drugs except for periods of about 200 ms during which the flow is retrograde at about one to 2 cm per second for about 150 ms. The retrograde flow will cause the infused fluid from the medial port to move in a retrograde manner over a distance of about 0.3 cm. The typical distance between ports on most central venous catheters is about 1 cm. The use of withdrawal rates that do not create enough suction to pull the glucose infusion across the port separation distance should be used when procuring blood samples for glucose measurement. Cross contamination can be prevented during blood withdrawal by interrupting the withdrawal of the sample during the inflation of the lung or at any point where cross-contamination is sufficiently likely. As noted previously, the large venous vessels in the thoracic cavity do not have valves, therefore flow is determined by pressure gradients. For the purposes of determining the presence of reverse flow, measurement of intravascular pressures or pressure changes can be beneficial. In the blood access circuit shown in FIG. 26, the two pressure transducers located on the pump console have the capability of measuring intravascular pressure. FIG. 27 shows the pressure tracing obtained during eight automated sample withdrawal, measurement, re-infusion and cleaning cycles. FIG. 28 illustrates the influence of ventilation during those periods of constant infusion typically referred to as KVO (“keep vein open”). During periods when one or more of the pumps are active the quality or information content of the intravascular pressure can be diminished by the influence of the withdrawal pumps. Due to this diminished signal it can be desirable to use a signal from the ventilator, or measured based on the ventilator, as the true signal of ventilator status. While this provides an assessment of ventilator status, it might not be an exact indicator of intravascular pressure due to a number of lags or pressure delays present in the body. For example, in the case of central venous catheter located in the abdomen, there can be an appreciable delay between the initiation of positive pressure ventilation and a corresponding pressure change at the catheter. Assuming that the catheter does not move appreciably, this delay can be quantified by examining the difference between the pressure response as measured from the ventilator and the corresponding pressure response measured in the vessel. This lag can be well-characterized during periods when the intravascular pressure signal is not corrupted by the withdrawal pumps. Such a period exists during KVO infusions. Multiple methodologies can be used to determine intravascular pressure and/or the correlation between intravascular pressure and the stage of ventilation. The following example embodiments include an example method for measuring the ventilator stage, concurrently measuring intravascular pressure and defining the associated lag.

In practice, it can be desirable to minimize the total time needed to withdraw the blood and eliminate any unwanted flow characteristics at the catheter tip due to the overall compliance of the circuit. These desired requirements can be achieved with responsive and active control of fluid flows, pressures, or a combination thereof. Four methods of interrupting flow for the purpose of anti-cross contamination controls have been identified: 1) a compliance isolation method, 2) a flow feedback method 3) a cascade pressure-flow feedback control method and 4) a pressure feedback method. For completeness of the description of operation, the block diagrams of these example circuits include direct measurement of the ventilator stage and include the determination of lag between the ventilator stage and the intravascular pressure change, although as described herein variations are possible.

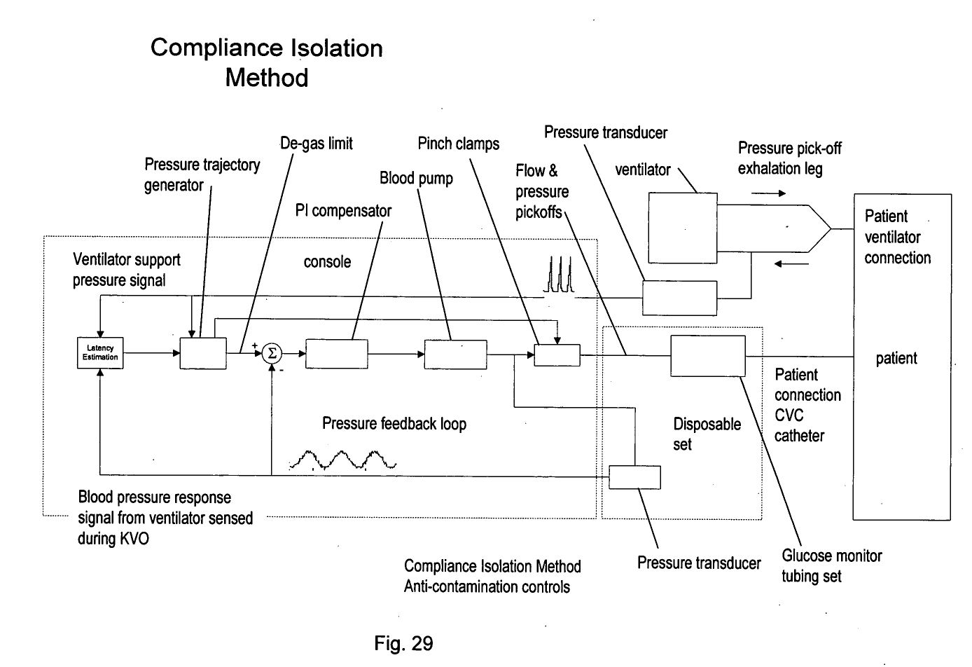

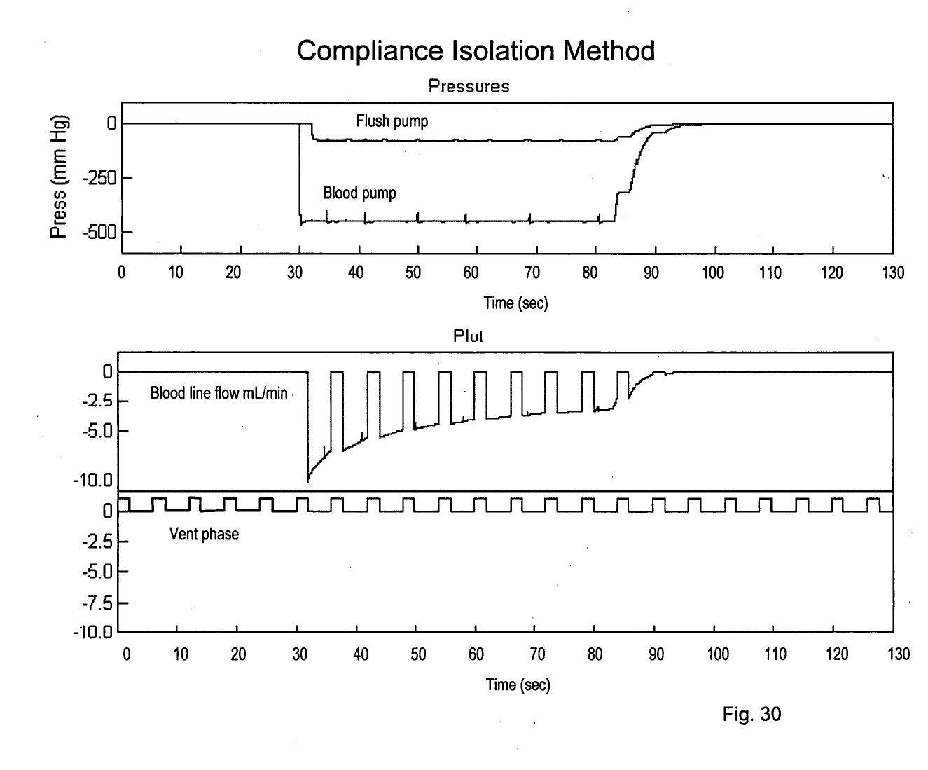

Compliance Isolation: The compliance isolation method provides anti-contamination control by using pinch valves that close fluid connections between the pump loop tubing and the sensor set at the blood optionally and optionally the flush pump, interrupting flow during the interval of lung inflation. This method works with the example pressure based withdrawal technique shown and prevents or minimizes flow reversal during the intervals of interruption by isolating the soft compliance of the pump loops from the stiffer portions of the sensor set. The pinch valves are activated immediately upon the signal of lung inflation and the pumps are allowed to continue operating at the pressure target with zero flow. Any alarms that would normally sense occlusions during the withdrawal can be deactivated during this interval. FIG. 29 shows a block diagram of the compliance isolation method. The pressure feedback loop comprises the sensor set blood line pressure transducer that provides a true measure of blood line pressure. This pressure is compared to the desired blood line pressure and the difference is used to control the blood pump through a control compensator that is structured and tuned to minimize this difference in transient and steady state conditions. With the pinch clamp open, the blood pump affects the flow and pressure in the tubing set. With the pinch clamp closed, the blood pump no longer affects either flow or pressure in the tubing set. Pressure between the blood pump and pinch valve are controlled to the desired pressure, but pressure and flow downstream of the pinch valve both drop to zero. FIG. 30 shows the simulated pressure and flow responses during a withdrawal where the compliance isolation method is used.

As shown in FIG. 29, the desired pressure target command shaping and timing can be determined according to a pressure reference trajectory generator that determines the latency between the ventilator pressure signal and ventilator induced pressure changes on the blood pressure measurement. These latencies can be determined during KVO operation and used to delay the command to stop flow with the pinch valves accordingly.

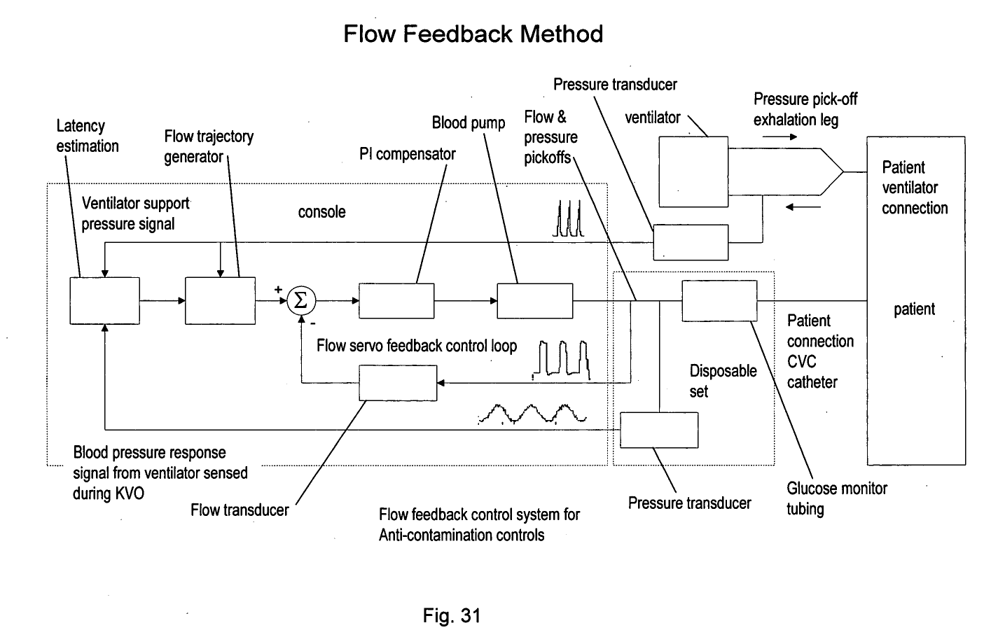

Flow Feedback Control: FIG. 31 illustrates a flow feedback method, using a flow sensor in the blood line to sense fluid flow which can be compared to a desired flow. The difference is fed to a controller which, when correctly tuned, commands the pump and minimizes the flow difference both during transient and steady states of flow. Thus the true flow will follow the desired flow. The flow feedback loop is operational all the time during the draw however the desired flow (command) is adjusted according to the state of lung inflation. During the state where the lung is not inflated, the desired flow is set to a constant flow target, and the withdrawal proceeds. When lung inflation is sensed, the desired flow is commanded to zero (or near zero) interrupting the withdrawal. The flow feedback loop stiffens the effective flow impedance of the sensor set. This results in a faster time constant in the flow response as compared to the sensor set without flow feedback where changes in flow are limited by the intrinsic compliance and resistance of the sensor set. Without flow feedback, the natural response of the sensor set causes flow withdrawal to continue even after the pump is stopped. With flow feedback the pump actually reverses direction to counteract this natural response and achieve zero flow in a more rapid manner.

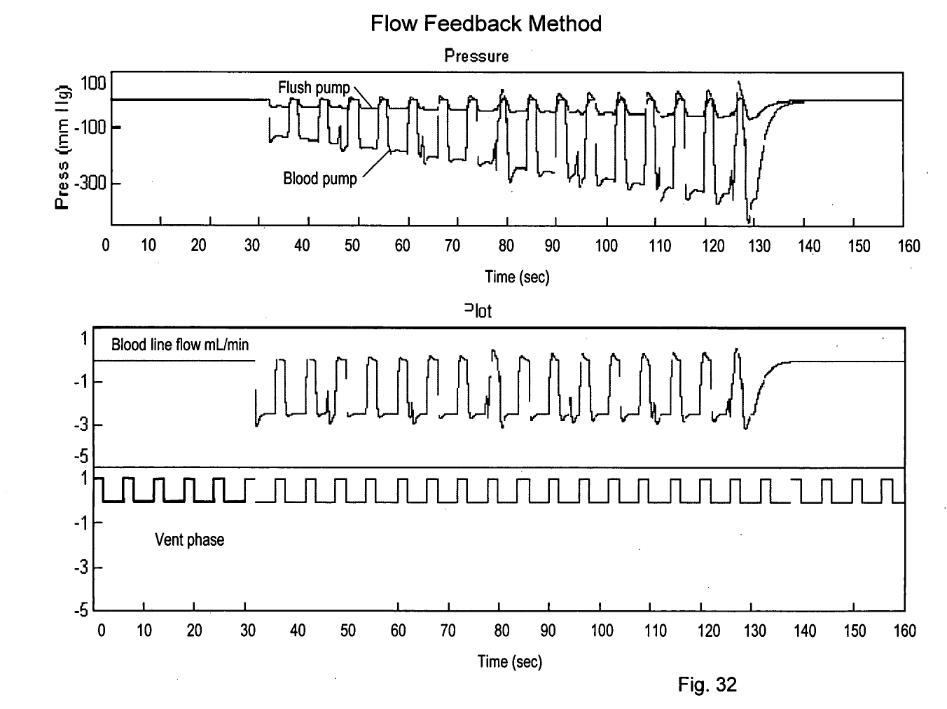

For this method to work properly, the desired flow target must be set at a value that does not cause pressure to exceed the pressure limit beyond which degassing of the fluids might be expected to occur. Pressure can increase as additional blood is drawn into the blood line so the flow target must be set so that pressure is maintained within the limit at the end of the draw. The desired flow target command shaping and timing are determined according to a flow reference trajectory generator that determines the latency between the ventilator pressure signal and ventilator induced pressure changes on the blood pressure measurement. These latencies are determined during KVO and used to delay the command to stop flow accordingly. FIG. 32 illustrates a simulated operation of the flow feedback control method during a withdrawal.

Cascaded Flow-Pressure Feedback Control: The cascade control method enhances the benefits of 1) maximum draw rate at a target negative upstream pressure which limits the de-gas rate of fluids during intervals where cross contamination is not expected, and 2) rapid deceleration of fluid flow rate to zero (or near zero) during intervals where cross contamination is expected. These benefits are achieved by using an inner, flow feedback control loop, and an outer, pressure feedback control loop. These inner and outer loops comprise the control cascade.

The inner flow feedback loop is operational all the time during the draw as well as draw interruptions, and the outer pressure feedback loop is only active between the flow interruptions. The inner flow feedback loop effectively stiffens the flow impedance of the sensor set. This results in a faster time constant in the flow response as compared to the sensor set without flow feedback where changes in flow rate are limited by the intrinsic compliance and resistance of the sensor set.

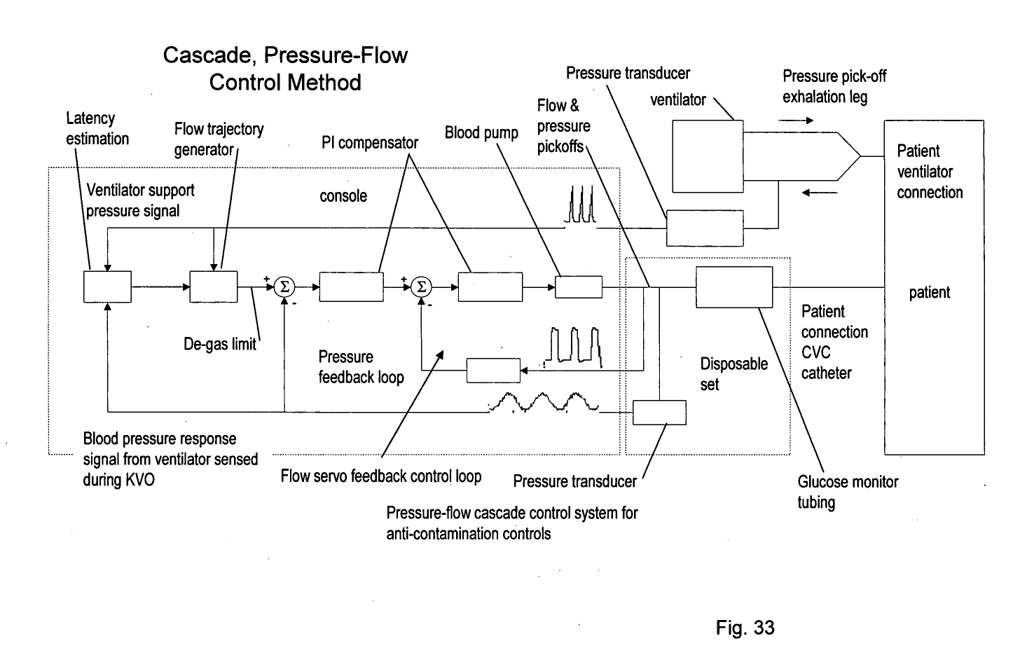

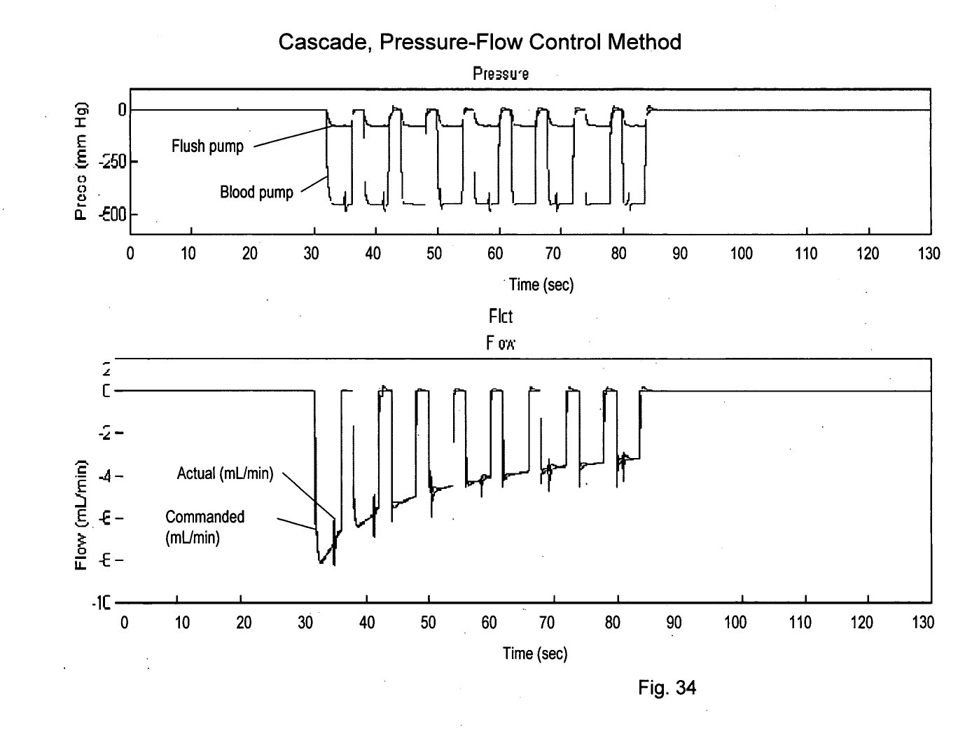

The outer pressure feedback loop provides the command signal to the inner flow feedback loop during the interval of lung deflation, where cross contamination is not expected to occur. The pressure loop targets a high negative pressure during that interval to maximize the draw rate however within a pressure constraint that prevents or minimizes degassing of the blood and maintenance fluid. During lung inflation the pressure controller is reset and held inactive with a command of zero flow to the inner flow loop. FIG. 33 illustrates, by block diagram, the cascade control method. FIG. 34 illustrates simulated operation of the cascade control method.

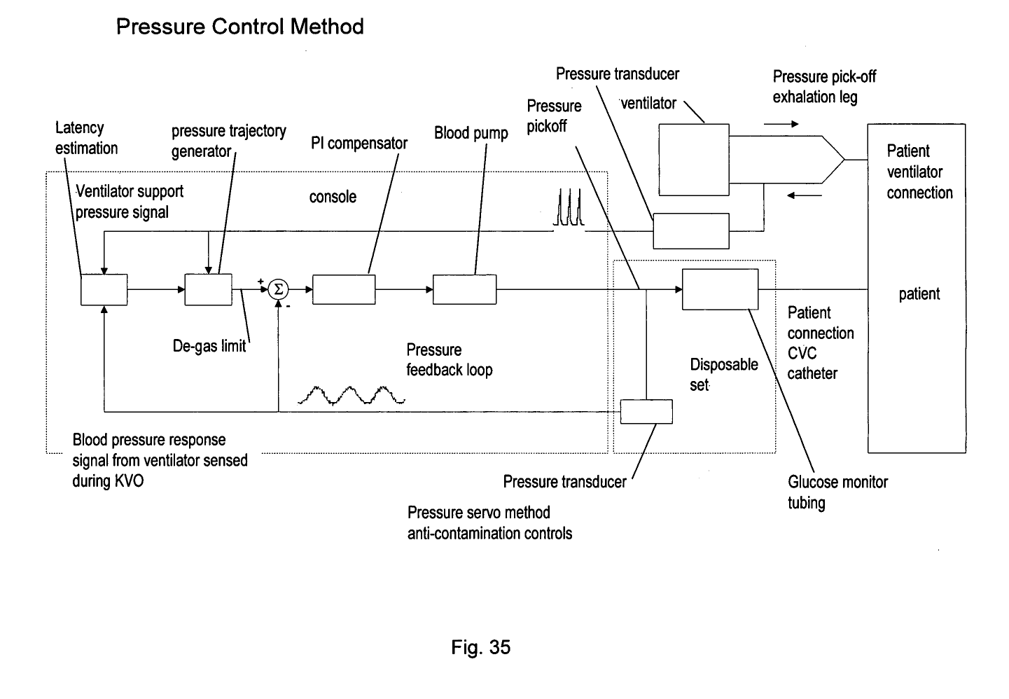

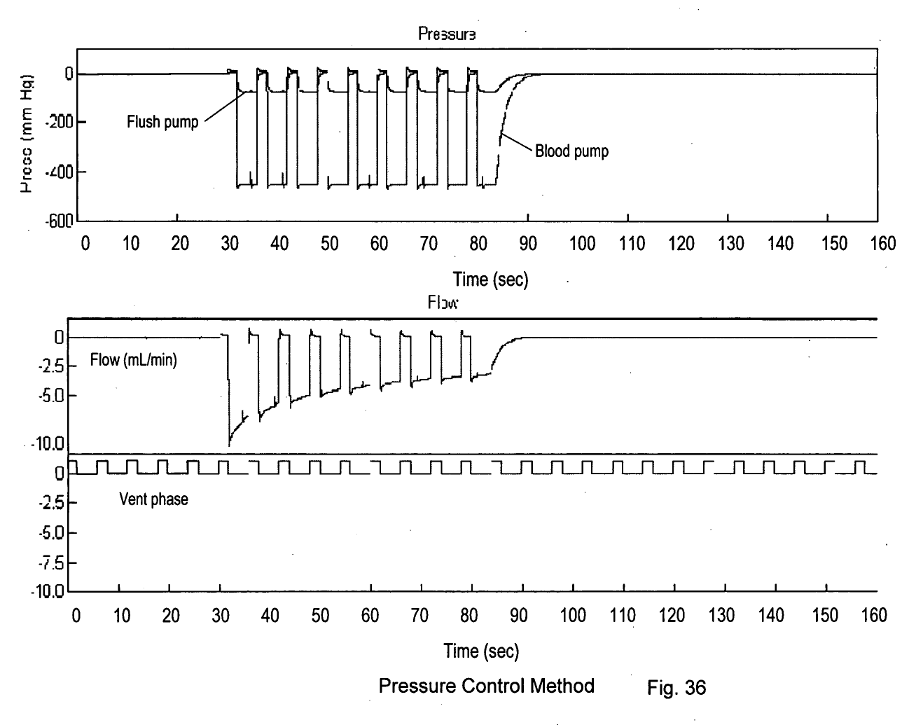

Pressure Feedback Control: The pressure feedback control method utilizes the same pressure feedback control servo used during the draw intervals for the intervals that interrupt withdrawal by substituting a slightly positive pressure target during these intervals. This results in an immediate reversal of the pump just after the draw which prevents reversal of flow during the interrupts and maintains a slight positive flow from the canula. FIG. 7 is a schematic block diagram of this approach where the pressure trajectory generator decides between the positive or negative pressure target based on the phase of ventilation. As described in the other methods, the pressure fluctuations observed from the blood pressure transducer are used to determine latency, if any, between pressure changes in the blood and those measured from the ventilator during KVO to delay action. FIG. 35 shows an example of the pressure feedback control method in simulation. FIG. 36 shows a simulator response using the pressure feedback control method.

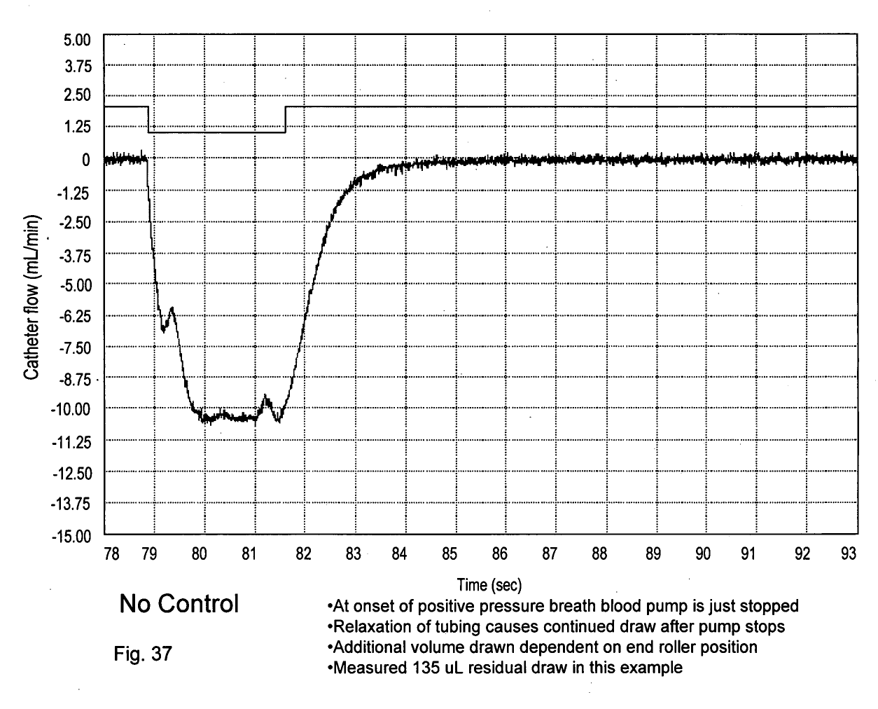

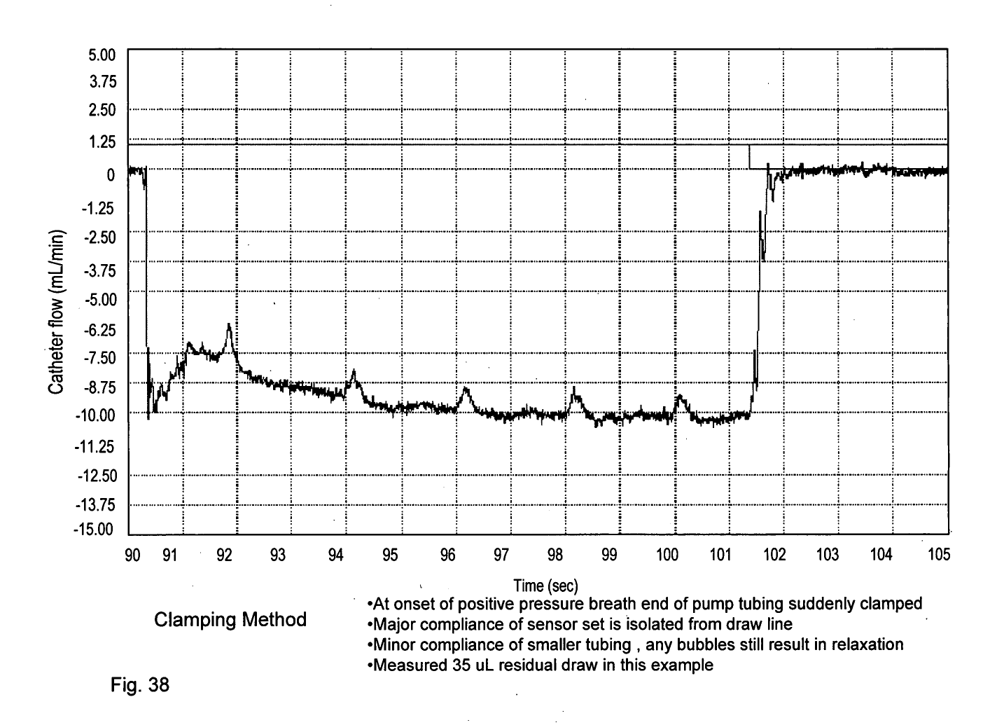

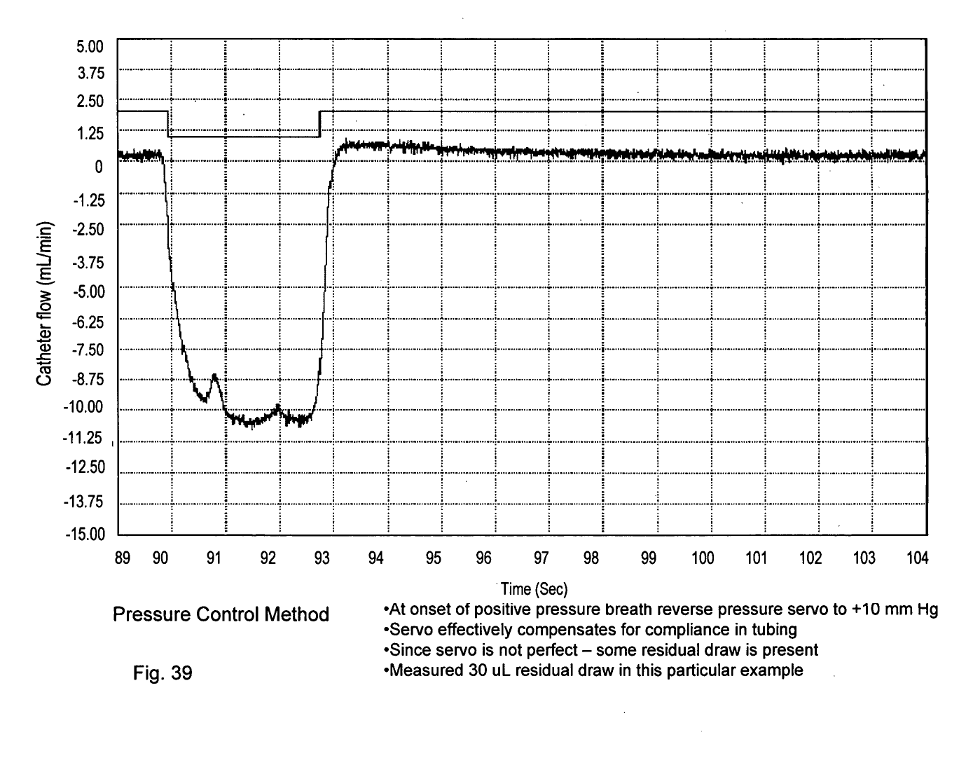

To further confirm the operational principles with respect to controlling flow in a blood access circuit, a simple confirmatory test was conducted in the laboratory. A blood access circuit and pumping mechanism as shown in FIG. 26 was utilized. At the end of the catheter, and ultrasonic flow sensor was placed for the recording of fluid flows. A simulated ventilator signal associated with inspiration was generated such that a stop flow or stop withdrawal signal was generated. The performance characteristics were then documented by the flow measurement system and response times were calculated. This proof of principle investigation sought to demonstrate the performance characteristics of: (1) no control, (2) the compliance isolation method and (3) pressure control method. The no control method was implemented by simply issuing a command to stop pumping via the peristaltic pumps. There is no active control to minimize any residual compliance artifacts in the circuit. In the case of the compliance isolation method the clamping methodology used a controlled hemostat. As can be seen in FIG. 37, the no control methodology can effectively start and stop the circuit but the residual compliance in the circuit results in an undesired continuation of the withdrawal for about 1.5 seconds and an additional unwanted withdrawal volume of approximately 135 uL. FIG. 38 shows the results from the isolation compliance method. The use of a clamp effectively stops flow when used below the compliant pump tubing. The unwanted withdrawal volume is now decreased to only 35 uL. FIG. 39 shows the implementation of the pressure control methodology. In this case the pump control servo mechanism was instructed to operate between −450 mm Hg and +10 mm Hg. As can be seen by the flow tracing this methodology has a very fast response time and results in very little unwanted withdrawal volume. Furthermore for the pressure control method, the set positive pressure during the period of lung inflation can be adjusted so that a small reverse flow is affected to entirely flush back any contaminated sample that might have entered the blood sampling line.

The particular sizes and equipment discussed above are cited merely to illustrate particular embodiments of the invention. It is contemplated that the use of the invention can involve components having different sizes and characteristics. It is intended that the scope of the invention be defined by the claims appended hereto.

What is claimed is:

1. A method for determining whether a measured value of a target analyte in blood measured at a first location is likely to be contaminated with an infusate infused at a second location, comprising comparing an indicator characteristic of the blood determined at a first time with the indicator characteristic of the blood determined at a second time, and evaluating the comparison against a metric.

2. A method as in claim 1, wherein the first and second times are during patient conditions similar to the patient conditions under which the target analyte is measured.

3. A method as in claim 1, wherein the metric reflects expected change in the indicator characteristic due only to physiological changes.

4. A method as in claim 1, wherein the indicator characteristic does not substantially change due to physiological processes but does change substantially in the presence of the infusate.

5. A method of determining whether patient conditions are likely to lead to contamination of a target blood analyte with an infusate, comprising comparing an indicator characteristic of the blood determined at a first time with the indicator characteristic of the blood determined at a second time, and evaluating the comparison against a metric.

6. A method as in claim 5, wherein the first and second times are during patient conditions similar to the patient conditions under which the target analyte is measured.

7. A method as in claim 5, wherein the metric reflects expected change in the indicator characteristic due only to physiological changes.

8. A method as in claim 5, wherein the indicator characteristic does not substantially change due to physiological processes but does change substantially in the presence of the infusate.

9. A method as in claim 1, wherein the indicator characteristic comprises measurement of the target analyte.

10. A method as in claim 9, wherein the target analyte is glucose.

11. A method as in claim 5, wherein the indicator characteristic comprises measurement of the target analyte.

12. A method as in claim 11, wherein the target analyte is glucose.

13. A method as in claim 1, wherein the indicator characteristic comprises measurement of a substance present in the infusate at a different concentration than in the blood.

14. A method as in claim 5, wherein the indicator characteristic comprises measurement of a substance present in the infusate at a different concentration than in the blood.

15. A method as in claim 1, wherein the indicator characteristic comprises measurement of a substance not present in the infusate.

16. A method as in claim 5, wherein the indicator characteristic comprises measurement of a substance not present in the infusate.

17. A method as in claim 13, wherein the substance is chosen from the group consisting of potassium, hemoglobin, albumin, triglycerides, serum proteins, or a combination thereof.

18. A method as in claim 14, wherein the substance is chosen from the group consisting of potassium, hemoglobin, albumin, triglycerides, serum proteins, or a combination thereof.

19. A method as in claim 1, wherein the indicator characteristic is chosen from the group consisting of optical scatter, electrical conductivity, absorbance spectrum, or a combination thereof.

20. A method as in claim 5, wherein the indicator characteristic is chosen from the group consisting of optical scatter, electrical conductivity, absorbance spectrum, or a combination thereof.

21. A method as in claim 1, wherein the patient is subject to mechanical ventilation, and the metric comprises a determination of whether the indicator characteristic changes in a manner correlated with the mechanical ventilation.

22. A method as in claim 5, wherein the patient is subject to mechanical ventilation, and the metric comprises a determination of whether the indicator characteristic changes in a manner correlated with the mechanical ventilation.

23. A method for measuring a target analyte in blood of a patient, comprising determining whether a measured value is likely to be contaminated according to the method of claim 1, and, if not, then measuring the target analyte in the blood and reporting the value.

24. A method for measuring a target analyte in a blood sample withdrawn from a patient, comprising withdrawing the blood sample, evaluating the blood sample for likely contamination according to the method of claim 5, and measuring and reporting the value of the target analyte if the evaluation indicates that contamination is unlikely.

25. A method as in claim 23, wherein measuring the value of the target analyte comprises measuring the target analyte concentration in blood from the response of the blood to incident light.

26. A method as in claim 24, wherein measuring the value of the target analyte comprises creating a portion of the blood sample having substantially no red blood cells, and measuring the target analyte concentration in the portion created.

27. A method for measuring a target analyte in blood of a patient, comprising determining whether a measured value is likely to be contaminated according to the method of claim 1, and measuring the target analyte in the blood wherein the measurement is adjusted based on the indicator characteristic.
